SEQUENCE LISTING (10 sequences)

SEQ ID NO: 1 - protein, 121 aa, unidentified
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 2 - protein, 108 aa, unidentified
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 3 - protein, 450 aa, unidentified
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 4 - protein, 214 aa, unidentified
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, unidentified
SSIMH

SEQ ID NO: 6 - protein, 17 aa, unidentified
YIKPYNDGTKYNEKFKG

SEQ ID NO: 7 - protein, 12 aa, unidentified
EGGNDYYDTMDY

SEQ ID NO: 8 - protein, 11 aa, unidentified
RASQDINSYLS

SEQ ID NO: 9 - protein, 7 aa, unidentified
RVNRLVD

SEQ ID NO: 10 - protein, 9 aa, unidentified
LQYDAFPYT

(19) United States
(12) Patent Application Publication
(10) Pub. No.: US 2025/0345449 A1
(43) Pub. Date: Nov. 13, 2025

(54) ANTI-CD123 IMMUNOCONJUGATES FOR THE TREATMENT OF BLASTIC PLASMACYTOID CELL NEOPLASM

(71) Applicant: ImmunoGen, Inc., North Chicago, IL (US)

(72) Inventor: Patrick Zweidler-McKay, North Chicago, IL (US)

(21) Appl. No.: 19/204,184

(22) Filed: May 9, 2025

Related U.S. Application Data
(60) Provisional application No. 63/645,614, filed on May 10, 2024.
(60) Provisional application No. 63/728,622, filed on Dec. 5, 2024.

(51) Int. Cl. A61K 47/68; A61K 31/138; A61K 31/167; A61K 31/573; A61K 35/28; A61P 35/00

(52) U.S. Cl. CPC A61K 47/6849; A61K 31/138; A61K 31/167; A61K 31/573; A61K 35/28; A61K 47/6803; A61P 35/00

(57) ABSTRACT

Methods and uses of immunoconjugates that bind to CD123 (e.g., pivekimab sunirine) in patients with blastic plasmacytoid dendritic cell neoplasm (BPDCN) are provided. Such immunoconjugates can be used, for example, to prepare patients with BPDCN for hematopoietic stem cell transplant, to achieve bone marrow remission in BPDCN patients with bone marrow involvement, and to achieve long-lasting complete remissions in patients with BPDCN as a result of a short treatment period.

### FIELD OF THE DISCLOSURE

The field of the disclosure generally relates to methods of treating Blastic Plasmacytoid Dendritic Cell Neoplasm (BPDCN), including frontline and relapsed/refractory BPDCN, in patients and methods for preparing patients with BPDCN for stem cell transplants using anti-CD123 immunoconjugates, e.g., pivekimab sunirine.

### CROSS-REFERENCE TO RELATED PATENT APPLICATIONS

This patent application claims the benefit of U.S. Provisional Patent Application No. 63/645,614, filed May 10, 2024, and U.S. Provisional Patent Application No. 63/728,622, filed Dec. 5, 2024. Each of the aforementioned patent applications is hereby incorporated by reference in its entirety.

### REFERENCE TO SEQUENCE LISTING SUBMITTED ELECTRONICALLY

The content of the electronically submitted sequence listing (Name: 6776-6902_Sequence_Listing.xml; Size: 10,668 bytes; and Date of Creation: May 8, 2025) filed with the application is incorporated herein by reference in its entirety.

### BACKGROUND

Blastic Plasmacytoid Dendritic Cell Neoplasm (BPDCN) is a rare, aggressive hematological malignancy derived from myeloid dendritic cell precursors with skin, lymph node, blood, central nervous system, and bone marrow involvement. BPDCN may arise de-novo or in the context of a prior or concomitant hematological malignancy (PCHM). Intense chemotherapy and tagraxofusp-erzs (tagraxofusp, ELZONRIS®) are standard-of-care treatment options in Europe and the United States. However, despite complete response (CR) rates of 47%-86% in frontline disease and median overall survival of approximately 12-24 months with these therapies, the majority of BPDCN patients will eventually relapse with no effective salvage treatment options. Hematopoietic stem cell transplants have also been considered for patients with BPDCN, but the toxicities associated with chemotherapies and tagraxofusp-erzs can render patients ineligible for such transplants. Therefore BPDCN is a serious disease with a high unmet need.

CD123 is the alpha-subunit of the interleukin-3 receptor (IL-3Ra). CD123 expression is low on normal hematopoietic stem cells (Testa et al., Biomark Res., 10; 2 (1): 4. (2014), Jordan et al., Leukemia, 14 (10): 1777-84 (2000)), but it is ubiquitously expressed in BPDCN blasts. An immunoconjugate that targets CD123 called IMGN632 or pivekimab sunirine (pvek) has been produced. This immunoconjugate contains a high-affinity anti-CD123 antibody, a cleavable linker, and an indolinobenzodiazepine pseudodimer (IGN) payload. The IGN payload alkylates DNA and causes single strand breaks without crosslinking. IGNs are designed to have high potency against tumor cells, while demonstrating less toxicity to normal marrow progenitors than other DNA-targeting payloads. Preclinically, pivekimab sunirine has demonstrated activity against BPDCN patient-derived xenografts. However, given the inability of currently available therapeutics to adequately treat BPDCN, there is a need for more effective interventions.

### SUMMARY OF THE DISCLOSURE

Provided herein is a method of preparing a subject with blastic plasmacytoid dendritic cell neoplasm (BPDCN) for a hematopoietic stem cell transplant (HSCT), the method comprising administering to the subject pivekimab sunirine.

Also provided herein is a method for treating a blastic plasmacytoid dendritic cell neoplasm (BPDCN) in a subject comprising (i) administering the subject pivekimab sunirine and (ii) subsequently administering a hematopoietic stem cell transplant (HSCT) to the subject.

Also provided herein is a method of treating a blastic plasmacytoid dendritic cell neoplasm (BPDCN) in a subject comprising administering a hematopoietic stem cell transplant (HSCT) to the subject, wherein the subject has previously been treated with pivekimab sunirine.

In some aspects provided herein, the HSCT is allogeneic. In some aspects provided herein, the HSCT is autologous. In some aspects provided herein, the HSCT is administered about 4 to about 8 weeks after the an administration of pivekimab sunirine.

In some aspects provided herein, the administration of the pivekimab sunirine produces a complete response (CR), CR (clinical) with minimal residual skin abnormality (CRc), CR (clinical) with partial hematological recovery (CRh), CR (clinical) with incomplete recovery (CRi), or a partial response. In some aspects, the administration of the pivekimab sunirine produces a complete response (CR), CR (clinical) with minimal residual skin abnormality (CRc), CR (clinical) with partial hematological recovery (CRh), or CR (clinical) with incomplete recovery (CRi). In some aspects, the administration of the pivekimab sunirine produces a partial response.

In some aspects provided herein, the time to a first response is about 4 months or less, about 3.5 months or less, about 2 months or less, or about 1.5 months or less.

In some aspects provided herein, the time to a complete response (CR), CR (clinical) with minimal residual skin abnormality (CRc), CR (clinical) with partial hematological recovery (CRh), or CR (clinical) with incomplete recovery (CRi) is about 5 months or less, about 4 months or less, about 3 months or less, about 2 months or less, or about 1.5 months or less.

Also provided herein is a method for treating a blastic plasmacytoid dendritic cell neoplasm (BPDCN) in a subject comprising administering to the subject pivekimab sunirine, wherein the time to a first response is about 4 months or less. In some aspects, the time to the first response is about 3.5 months or less. In some aspects, the time to the first response is about 2 months or less. In some aspects, the time to the first response is about 1.5 months or less.

Also provided herein is a method for treating a blastic plasmacytoid dendritic cell neoplasm (BPDCN) in a subject comprising administering to the subject pivekimab sunirine, wherein the time to a complete response (CR), CR (clinical) with minimal residual skin abnormality (CRc), CR (clinical) with partial hematological recovery (CRh), or CR (clinical) with incomplete recovery (CRi) is about 5 months or less. In some aspects, the time to the complete response (CR), CR (clinical) with minimal residual skin abnormality (CRc), CR (clinical) with partial hematological recovery (CRh), or CR (clinical) with incomplete recovery (CRi) is about 4 months or less. In some aspects, the time to the complete response (CR), CR (clinical) with minimal residual skin abnormality (CRc), CR (clinical) with partial hematological recovery (CRh), or CR (clinical) with incomplete recovery (CRi) is about 3 months or less. In some aspects, the time to the complete response (CR), CR (clinical) with minimal residual skin abnormality (CRc), CR (clinical) with partial hematological recovery (CRh), or CR (clinical) with incomplete recovery (CRi) is about 2 months or less. In some aspects, the time to the complete response (CR), CR (clinical) with minimal residual skin abnormality (CRc), CR (clinical) with partial hematological recovery (CRh), or CR (clinical) with incomplete recovery (CRi) is about 1.5 months or less.

In some aspects provided herein, the subject has a response with a duration of at least 6 months, at least 7 months, at least 8 months, at least 9 months, at least 10 months, at least 11 months or at least 12 months.

In some aspects provided herein, the subject has a complete response (CR), CR (clinical) with minimal residual skin abnormality (CRc), CR (clinical) with partial hematological recovery (CRh), or CR (clinical) with incomplete recovery (CRi) with a duration of at least 8 months, at least 9 months, or at least 10 months.

Also provided herein is a method for treating a blastic plasmacytoid dendritic cell neoplasm (BPDCN) in a subject comprising administering to the subject pivekimab sunirine, wherein the subject has a response to the administration, wherein the duration of the response is at least 6 months. In some aspects, the duration of the response is at least 7 months. In some aspects, the duration of the response is at least 8 months. In some aspects, the duration of the response is at least 9 months. In some aspects, the duration of the response is at least 10 months. In some aspects, the duration of the response is at least 11 months. In some aspects, the duration of the response is at least 12 months.

Also provided herein is a method for treating a blastic plasmacytoid dendritic cell neoplasm (BPDCN) in a subject comprising administering to the subject pivekimab sunirine, wherein the subject has a complete response (CR), CR (clinical) with minimal residual skin abnormality (CRc), CR (clinical) with partial hematological recovery (CRh), or CR (clinical) with incomplete recovery (CRi) to the administration and the duration of the complete response (CR), CR (clinical) with minimal residual skin abnormality (CRc), CR (clinical) with partial hematological recovery (CRh), or CR (clinical) with incomplete recovery (CRi) is at least 8 months. In some aspects, the duration of the complete response (CR), CR (clinical) with minimal residual skin abnormality (CRc), CR (clinical) with partial hematological recovery (CRh), or CR (clinical) with incomplete recovery (CRi) is at least 9 months. In some aspects, the duration of the complete response (CR), CR (clinical) with minimal residual skin abnormality (CRc), CR (clinical) with partial hematological recovery (CRh), or CR (clinical) with incomplete recovery (CRi) is at least 10 months.

In some aspects provided herein, the administration of pivekimab sunirine achieves bone marrow remission in the subject.

Also provided herein is a method for achieving bone marrow remission in a subject with blastic plasmacytoid dendritic cell neoplasm (BPDCN) with bone marrow involvement, the method comprising administering to the pivekimab sunirine.

In some aspects provided herein, the method further comprises administering to the subject a hematopoietic stem cell transplant (HSCT). In some aspects provided herein, the method does not further comprise administering to the subject a hematopoietic stem cell transplant (HSCT).

In some aspects provided herein, the subject has involvement of skin, bone marrow, lymph node, viscera, or a combination thereof in the BPDCN. In some aspects, the administration of pivekimab sunirine improves symptoms in the skin, bone marrow, lymph node, viscera, or a combination thereof.

In some aspects provided herein, the subject has involvement of skin, bone marrow, lymph node, or a combination thereof in the BPDCN. In some aspects, the administration of pivekimab sunirine improves symptoms in the skin, bone marrow, lymph node, or a combination thereof.

In some aspects provided herein, the subject has received tagraxofusp prior to the administration.

In some aspects provided herein, the subject has previously been treated with venetoclax; a hypomethylating agent; cyclophosphamide, vincristine sulfate, and prednisone (CVP); and/or steroids.

In some aspects provided herein, the subject has previously been treated with cyclophosphamide, vincristine sulfate, doxorubicin hydrochloride, methotrexate, cytarabine, and dexamethasone (HyperCVAD); fludarabine, high-dose cytarabine, and G-CSF (FLAG) and/or cyclophosphamide, vincristine and doxorubicin (CHOP).

In some aspects provided herein, the subject requires red blood cell and/or platelet transfusions prior to the administration of pivekimab sunirine. In some aspects, the subject becomes independent of the red blood cell and/or platelet transfusions for at least 28 consecutive days. In some aspects, the subject becomes independent of the red blood cell and/or platelet transfusions for at least 56 consecutive days.

In some aspects provided herein, the subject received a stem cell transplant prior to the administration of pivekimab sunirine. In some aspects, the stem cell transplant was received at least 120 days prior to the administration of pivekimab sunirine. In some aspects, the previous stem cell transplant was allogeneic. In some aspects, the previous stem cell transplant was autologous.

In some aspects provided herein, the BPDCN is relapsed or refractory (R/R) BPDCN. In some aspects, the BPDCN is a relapsed BPDCN. In some aspects, the BPDCN is a refractory BPDCN.

In some aspects provided herein, the patient has received 1-3 prior systemic therapies.

In some aspects provided herein, the subject received at least one prior line of therapy, at least two prior lines of therapy, or at least three prior lines of systemic therapy.

In some aspects provided herein, the administration of pivekimab sunirine is a frontline therapy. In some aspects, the BPDCN is de novo. In some aspects, the subject has no prior or concomitant hematological malignancy (PCHM). In some aspects, the subject has a prior or concomitant hematological malignancy (PCHM). In some aspects, the PCHM does not require immediate therapy. In some aspects, the PCHM is in remission and the subject has completed all therapies for the PCHM at least 6 months prior to the administration of pivekimab sunirine.

In some aspects provided herein, the subject has not received tagraxofusp prior to the administration.

In some aspects provided herein, the subject has not received immunosuppressive treatment for at least 14 days prior to the administration of pivekimab sunirine.

In some aspects provided herein, the subject has not received a systemic anti-cancer therapy for at least 14 days prior to the administration of pivekimab sunirine.

In some aspects provided herein, the subject received a local therapy at least 14 days prior to the administration of pivekimab sunirine. In some aspects, the therapy was radiotherapy.

In some aspects provided herein, the subject has a history of central nervous system (CNS) involvement in the BPDCN. In some aspects, the CNS involvement was treated locally and the subject has had at least one lumbar puncture with no evidence of CNS disease.

In some aspects provided herein, the treatment further comprises a CNS prophylactic treatment. In some aspects, the CNS prophylactic treatment comprises intrathecal chemotherapy.

In some aspects provided herein, the method further comprises administration of ursodeoxycholic acid.

In some aspects provided herein, the subject has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

In some aspects provided herein, the pivekimab sunirine is administered intravenously.

In some aspects provided herein, the pivekimab sunirine is administered in an infusion having a duration of no more than 30 minutes. In some aspects provided herein, the pivekimab sunirine is administered in an infusion having a duration of about 15 to 30 minutes. In aspects, the pivekimab sunirine is administered at an infusion rate of from about 0.8 mL/min to about 1.7 mL/min. In some aspects, the pivekimab sunirine is administered at an infusion rate of about 0.8 mL/min (about 50 mL/hour or about 0.165 mg/min) for the first 30 minutes. In aspects, the infusion rate may be increased to about 1.7 mL/min (100 mL/hour or 0.33 mg/min). In aspects, the infusion rate may be increased to about 1.7 mL/min (100 mL/hour or 0.33 mg/min), if well tolerated whereby no infusion-related reactions are observed in the patient. In aspects, the pivekimab sunirine is not administered by IV push.

In some aspects provided herein, the pivekimab sunirine is administered at a dose of about 0.045 mg/kg.

In some aspects provided herein, the pivekimab sunirine is administered once in a 21-day cycle.

In some aspects provided herein, the pivekimab sunirine is administered for one cycle. In some aspects provided herein, the pivekimab sunirine is administered for about 2 to about 4 cycles. In some aspects provided herein, the pivekimab sunirine is administered for more than one cycle, optionally wherein the administration is for at least 2 cycles, at least 3 cycles, at least 4 cycles, at least 5 cycles, at least 6 cycles, at least 7 cycles, at least 8 cycles, at least 9 cycles, or at least 10 cycles or wherein the administration is for about 2-4 cycles, about 2-6 cycles, about 2-8 cycles, about 2-10 cycles, or about 2-12 cycles.

In some aspects provided herein, the method further comprises administering a reduced dose of the pivekimab sunirine after a dose-limiting toxicity has occurred in the subject and has been reduced to baseline or ≤Grade 2.

In some aspects provided herein, the pivekimab sunirine is further administered as a maintenance therapy. In some aspects, the maintenance therapy comprises administering the pivekimab sunirine once in a 21-day cycle.

In some aspects provided herein, CD123 has been detected in a sample obtained from the BPDCN prior to the administration of pivekimab sunirine. In some aspects, the CD123 was detected using flow cytometry or immunohistochemistry.

In some aspects provided herein, the method further comprises detecting CD123 in a sample obtained from the BPDCN prior to the administration of pivekimab sunirine.

In some aspects provided herein, at least 80% of cells in the BPDCN express CD123.

In some aspects provided herein, CD123 has been detected in at least 80% of cells in a sample obtained from the BPDCN prior to the administration of pivekimab sunirine.

In some aspects provided herein, the method further comprises detecting CD123 in at least 80% of cells in a sample obtained from the BPDCN prior to the administration of pivekimab sunirine.

In some aspects provided herein, the BPDCN expresses multidrug resistance 1 (MDR1).

In some aspects provided herein, the BPDCN expresses P-glycoprotein (P-gp).

In some aspects provided herein, the subject has an absolute neutrophil count of greater than 500/μL.

In some aspects provided herein, the subject has been pretreated with a premedication prior to administration of the pivekimab sunirine, optionally wherein the premedication is a corticosteroid. In some aspects, premedications administered prior to the administration of pivekimab sunirine may be one or more of diphenhydramine, acetaminophen, paracetamol, dexamethasone, or a combination thereof.

In some aspects provided herein, the method further comprises pretreating the subject with a premedication or corticosteroid prior to administration of the pivekimab sunirine, optionally wherein the premedication or corticosteroid is diphenhydramine, acetaminophen, paracetamol, dexamethasone, or a combination thereof.

In some aspects provided herein, pivekimab sunirine is administered intravenously at a dose of about 0.045 mg/kg once in a 21-day cycle. In some aspects, the BPDCN is a frontline BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 5 months of the first administration, and wherein the duration of the CR, CRc, CRh, or CRi is at least 12 months. In some aspects, the BPDCN is a frontline BPDCN, and wherein the administration achieves a complete response (CR) or CR (clinical) with minimal residual skin abnormality (CRc), and wherein the duration of the CR or CRc is at least 8 months. In some aspects, the BPDCN is a frontline BPDCN, and wherein the administration achieves a complete response (CR) or CR (clinical) with minimal residual skin abnormality (CRc), and wherein the duration of the CR or CRc is at least 9 months. In some aspects, the subject has frontline BPDCN with no prior or concomitant hematological malignancy (PCHM). In some aspects, the BPDCN is a R/R BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 2 months of the first administration, and wherein the duration of the CR, CRc, CRh, or CRi is at least 6 months or at least 7 months.

Also provided herein is the use of pivekimab sunirine in any method provided herein or in the preparation for a medicament for use in any method provided herein.

In some embodiments (E1 to E141), the disclosure provides:

E1. A method of preparing a subject with blastic plasmacytoid dendritic cell neoplasm (BPDCN) for a hematopoietic stem cell transplant (HSCT), the method comprising administering to the subject a safe and effective amount of pivekimab sunirine.

E2. A method for treating a blastic plasmacytoid dendritic cell neoplasm (BPDCN) in a subject comprising (i) administering the subject a safe and effective amount of pivekimab sunirine and (ii) subsequently administering a hematopoietic stem cell transplant (HSCT) to the subject.

E3. A method of treating a blastic plasmacytoid dendritic cell neoplasm (BPDCN) in a subject comprising administering a hematopoietic stem cell transplant (HSCT) to the subject, wherein the subject has previously been treated with pivekimab sunirine.

E4. The method of any one of E1-E3, wherein the HSCT is allogeneic.

E5. The method of any one of E1-E3, wherein the HSCT is autologous.

E6. The method of any one of E1-E5, wherein the HSCT is administered about 4 to about 8 weeks after the administration of pivekimab sunirine.

E7. The method of any one of E1-E6, wherein the administration of the pivekimab sunirine produces a complete response (CR), CR (clinical) with minimal residual skin abnormality (CRc), CR (clinical) with partial hematological recovery (CRh), CR (clinical) with incomplete recovery (CRi), or a partial response.

E8. The method of E7, wherein the administration of the pivekimab sunirine produces a complete response (CR), CR (clinical) with minimal residual skin abnormality (CRc), CR (clinical) with partial hematological recovery (CRh), or CR (clinical) with incomplete recovery (CRi).

E9. The method of E7, wherein the administration of the pivekimab sunirine produces a partial response.

E10. The method of any one of E1-E9, wherein the time to a first response is about 4 months or less, about 3.5 months or less, about 2 months or less, or about 1.5 months or less.

E11. The method of any one of E1-E10, wherein the time to a complete response (CR), CR (clinical) with minimal residual skin abnormality (CRc), CR (clinical) with partial hematological recovery (CRh), or CR (clinical) with incomplete recovery (CRi) is about 5 months or less, about 4 months or less, about 3 months or less, about 2 months or less, or about 1.5 months or less.

E12. A method for treating a blastic plasmacytoid dendritic cell neoplasm (BPDCN) in a subject comprising administering to the subject a safe and effective amount of pivekimab sunirine, wherein the time to a first response is about 4 months or less.

E13. The method of E12, wherein the time to the first response is about 3.5 months or less.

E14. The method of E13, wherein the time to the first response is about 2 months or less.

E15. The method of E14, wherein the time to the first response is about 1.5 months or less.

E16. A method for treating a blastic plasmacytoid dendritic cell neoplasm (BPDCN) in a subject comprising administering to the subject a safe and effective amount of pivekimab sunirine, wherein the time to a complete response (CR), CR (clinical) with minimal residual skin abnormality (CRc), CR (clinical) with partial hematological recovery (CRh), or CR (clinical) with incomplete recovery (CRi) is about 5 months or less.

E17. The method of E16, wherein the time to the complete response (CR), CR (clinical) with minimal residual skin abnormality (CRc), CR (clinical) with partial hematological recovery (CRh), or CR (clinical) with incomplete recovery (CRi) is about 4 months or less.

E18. The method of E17, wherein the time to the complete response (CR), CR (clinical) with minimal residual skin abnormality (CRc), CR (clinical) with partial hematological recovery (CRh), or CR (clinical) with incomplete recovery (CRi) is about 3 months or less.

E19. The method of E18, wherein the time to the complete response (CR), CR (clinical) with minimal residual skin abnormality (CRc), CR (clinical) with partial hematological recovery (CRh), or CR (clinical) with incomplete recovery (CRi) is about 2 months or less.

E20. The method of E19, wherein the time to the complete response (CR), CR (clinical) with minimal residual skin abnormality (CRc), CR (clinical) with partial hematological recovery (CRh), or CR (clinical) with incomplete recovery (CRi) is about 1.5 months or less.

E21. The method of any one of E1-E20, wherein the subject has a response with a duration of at least 6 months, at least 7 months, at least 8 months, at least 9 months, at least 10 months, at least 11 months or at least 12 months.

E22. The method of any one of E1-E21, wherein the subject has a complete response (CR), CR (clinical) with minimal residual skin abnormality (CRc), CR (clinical) with partial hematological recovery (CRh), or CR (clinical) with incomplete recovery (CRi) with a duration of at least 8 months, at least 9 months, or at least 10 months.

E23. A method for treating a blastic plasmacytoid dendritic cell neoplasm (BPDCN) in a subject comprising administering to the subject a safe and effective amount of pivekimab sunirine, wherein the subject has a complete response (CR), CR (clinical) with minimal residual skin abnormality (CRc), CR (clinical) with partial hematological recovery (CRh), or CR (clinical) with incomplete recovery (CRi) to the administration and the duration of the complete response (CR), CR (clinical) with minimal residual skin abnormality (CRc), CR (clinical) with partial hematological recovery (CRh), or CR (clinical) with incomplete recovery (CRi), wherein the duration of the response is at least 6 months.

E24. The method of E23, wherein the duration of the response is at least 7 months.

E25. The method of E23, wherein the duration of the response is at least 8 months.

E26. The method of E24, wherein the duration of the response is at least 9 months.

E27. The method of E25, wherein the duration of the response is at least 10 months.

E28. The method of E25, wherein the duration of the response is at least 11 months.

E29. The method of E25, wherein the duration of the response is at least 12 months.

E30. A method for treating a blastic plasmacytoid dendritic cell neoplasm (BPDCN) in a subject comprising administering to the subject a safe and effective amount of pivekimab sunirine, wherein the subject has a complete response (CR), CR (clinical) with minimal residual skin abnormality (CRc), CR (clinical) with partial hematological recovery (CRh), or CR (clinical) with incomplete recovery (CRi) to the administration and the duration of the complete response (CR), CR (clinical) with minimal residual skin abnormality (CRc), CR (clinical) with partial hematological recovery (CRh), or CR (clinical) with incomplete recovery (CRi) is at least 8 months.

E31. The method of E29, wherein the duration of the complete response (CR), CR (clinical) with minimal residual skin abnormality (CRc), CR (clinical) with partial hematological recovery (CRh), or CR (clinical) with incomplete recovery (CRi) is at least 9 months.

E32. The method of E30, wherein the duration of the complete response (CR), CR (clinical) with minimal residual skin abnormality (CRc), CR (clinical) with partial hematological recovery (CRh), or CR (clinical) with incomplete recovery (CRi) is at least 10 months.

E33. The method of any one of E1-E31, wherein the administration of pivekimab sunirine achieves bone marrow remission in the subject.

E34. A method for achieving bone marrow remission in a subject with blastic plasmacytoid dendritic cell neoplasm (BPDCN) with bone marrow involvement, the method comprising administering to the subject a safe and effective amount of pivekimab sunirine.

E35. The method of any one of E12-E34, wherein the method further comprises administering to the subject a hematopoietic stem cell transplant (HSCT).

E36. The method of any one of E12-E34, wherein the method does not further comprise administering to the subject a hematopoietic stem cell transplant (HSCT).

E37. The method of any one of E1-E35, wherein the subject has involvement of skin, bone marrow, lymph node, viscera, or a combination thereof in the BPDCN.

E38. The method of E36, wherein the administration of pivekimab sunirine improves symptoms in the skin, bone marrow, lymph node, viscera, or a combination thereof.

E39. The method of any one of E1-E35, wherein the subject has involvement of skin, bone marrow, lymph node, or a combination thereof in the BPDCN.

E40. The method of E38, wherein the administration of pivekimab sunirine improves symptoms in the skin, bone marrow, lymph node, or a combination thereof.

E41. The method of any one of E1-E39, wherein the subject has received tagraxofusp prior to the administration of pivekimab sunirine.

E42. The method of any one of E1-E40, wherein the subject has previously been treated with venetoclax; a hypomethylating agent; cyclophosphamide, vincristine sulfate, and prednisone (CVP); and/or steroids.

E43. The method of any one of E1-E41, wherein the subject has previously been treated with cyclophosphamide, vincristine sulfate, doxorubicin hydrochloride, methotrexate, cytarabine, and dexamethasone (HyperCVAD); fludarabine, high-dose cytarabine, and G-CSF (FLAG); and/or cyclophosphamide, vincristine and doxorubicin (CHOP).

E44. The method of any one of E1-E42, wherein the subject requires red blood cell and/or platelet transfusions prior to the administration of pivekimab sunirine.

E45. The method of E44, wherein the subject becomes independent of the red blood cell and/or platelet transfusions for at least 28 consecutive days.

E46. The method of E44, wherein the subject becomes independent of the red blood cell and/or platelet transfusions for at least 56 consecutive days.

E47. The method of any one of E1-E46, wherein the subject received a stem cell transplant prior to the administration of pivekimab sunirine.

E48. The method of E47, wherein the stem cell transplant was received at least 120 days prior to the administration of pivekimab sunirine.

E49. The method of E47 or E48, wherein the previous stem cell transplant was allogeneic.

E50. The method of E47 or E48, wherein the previous stem cell transplant was autologous.

E51. The method of any one of E1-E50, wherein the BPDCN is relapsed or refractory (R/R) BPDCN.

E52. The method of E51, wherein the BPDCN is a relapsed BPDCN.

E53. The method of E51, wherein the BPDCN is a refractory BPDCN.

E54. The method of any one of E1-E53, wherein the patient has received 1-3 prior systemic therapies.

E55. The method of any one of E1-E53, wherein the subject received at least one prior line of therapy, at least two prior lines of therapy, or at least three prior lines of systemic therapy.

E56. The method of any one of E1-E46, wherein the administration of pivekimab sunirine is a frontline therapy.

E57. The method of any one of E1-E46 or E56, wherein the BPDCN is de novo.

E58. The method of E55, wherein the subject has a prior or concomitant hematological malignancy (PCHM).

E59. The method of E58, wherein the PCHM does not require immediate therapy.

E60. The method of any one of E58 or E59, wherein the PCHM is in remission and the subject has completed all therapies for the PCHM at least 6 months prior to the administration of pivekimab sunirine.

E61. The method of any one of E1-E60 wherein the subject has not received tagraxofusp prior to the administration of pivekimab sunirine.

E62. The method of any one of E1-E60, wherein the subject has not received immunosuppressive treatment for at least 14 days prior to the administration of pivekimab sunirine.

E63. The method of any one of E1-E61, wherein the subject has not received a systemic anti-cancer therapy for at least 14 days prior to the administration of pivekimab sunirine.

E64. The method of any one of E1-E62, wherein the subject received a local therapy at least 14 days prior to the administration of pivekimab sunirine.

E65. The method of E63, wherein the therapy was radiotherapy.

E66. The method of any one of E1-E64, wherein the subject has a history of central nervous system (CNS) involvement in the BPDCN.

E67. The method of E65, wherein the CNS involvement was treated locally and the subject has had at least one lumbar puncture with no evidence of CNS disease.

E68. The method of any one of E1-E66, wherein the treatment further comprises a CNS prophylactic treatment.

E69. The method of E67, wherein the CNS prophylactic treatment comprises intrathecal chemotherapy.

E70. The method of any one of E1-E68, wherein the method further comprises administration of ursodeoxycholic acid.

E71. The method of any one of E1-E69, wherein the subject has liver enzymes less than or equal to 2.5×the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

E72. The method of any one of E1-E71, wherein the pivekimab sunirine is administered intravenously.

E73. The method of any one of E1-E72, wherein the pivekimab sunirine is administered by a controlled rate of infusion.

E74. The method of any one of E1-E73, wherein the pivekimab sunirine is administered in an infusion having a duration of no more than 30 minutes.

E75. The method of any one of E1-E73, wherein the pivekimab sunirine is administered in an infusion having a duration of about 15 to 30 minutes.

E76. The method of any one of E73-E75, wherein the controlled rate of infusion is from about 0.8 mL/min to about 1.7 mL/min.

E77. The method of any one of E73-E75, wherein the controlled rate of infusion is about 0.8 mL/min (about 50 mL/hour or about 0.165 mg/min) for the first 30 minutes.

E78. The method of any one of E73-E76, wherein the controlled rate of infusion is increased to about 1.7 mL/min.

E79. The method of any one of E1-E78, wherein the pivekimab sunirine is administered at a dose of about 0.045 mg/kg.

E80. The method of any one of E1-E79, wherein the pivekimab sunirine is administered once in a 21-day cycle.

E81. The method of any one of E1-E79, wherein the pivekimab sunirine is administered for one cycle.

E82. The method of any one of E1-E79, wherein the pivekimab sunirine is administered for about 2 to about 4 cycles.

E83. The method of any one of E1-E79, wherein the pivekimab sunirine is administered for more than one cycle, optionally wherein the administration is for at least 2 cycles, at least 3 cycles, at least 4 cycles, at least 5 cycles, at least 6 cycles, at least 7 cycles, at least 8 cycles, at least 9 cycles, or at least 10 cycles or wherein the administration is for about 2-4 cycles, about 2-6 cycles, about 2-8 cycles, about 2-10 cycles, or about 2-12 cycles.

E84. The method of any one of E1-E83, wherein the method further comprises administering a reduced dose of the pivekimab sunirine after a dose-limiting toxicity has occurred in the subject and has been reduced to baseline or ≤Grade 2.

E85. The method of any one of E1-E84, wherein the pivekimab sunirine is further administered as a maintenance therapy.

E86. The method of E85, wherein the maintenance therapy comprises administering the pivekimab sunirine once in a 21-day cycle.

E87. The method of any one of E1-E86, wherein CD123 has been detected in a sample obtained from the BPDCN prior to the administration of pivekimab sunirine.

E88. The method of E87, wherein the CD123 was detected using flow cytometry or immunohistochemistry.

E89. The method of any one of E1-E88, further comprising detecting CD123 in a sample obtained from the BPDCN prior to the administration of pivekimab sunirine.

E90. The method of any one of E1-E89, wherein at least 80% of cells in the BPDCN express CD123.

E91. The method of any one of E1-E90, wherein CD123 has been detected in at least 80% of cells in a sample obtained from the BPDCN prior to the administration of pivekimab sunirine.

E92. The method of any one of E1-E90, further comprising detecting CD123 in at least 80% of cells in a sample obtained from the BPDCN prior to the administration of pivekimab sunirine.

E93. The method of any one of E1-E92, wherein the BPDCN expresses multidrug resistance 1 (MDR1).

E94. The method of any one of E1-E93, wherein the BPDCN expresses P-glycoprotein (P-gp).

E95. The method of any one of E1-E94, wherein the subject has an absolute neutrophil count of greater than 500/μL.

E96. The method of any one of E1-E95, wherein the subject has been pretreated with a premedication or corticosteroid prior to administration of the pivekimab sunirine, optionally wherein the premedication or corticosteroid is diphenhydramine, acetaminophen, paracetamol, dexamethasone, or a combination thereof.

E97. The method of any one of E1-E95, further comprising pretreating the subject with a premedication or corticosteroid prior to administration of the pivekimab sunirine, optionally wherein the premedication or corticosteroid is diphenhydramine, acetaminophen, paracetamol, dexamethasone, or a combination thereof.

E98. The method of any one of E96-E97, wherein the corticosteroid is administered to a patient on the day prior and once, prior to and on the same day as administering an anti-CD123 immunoconjugate, e.g., pivekimab sunirine, to the patient.

E99. The method of any one of E96-E97, wherein the corticosteroid is administered to a patient twice on the day prior and once, prior to and on the same day as administering an anti-CD123 immunoconjugate, e.g., pivekimab sunirine, to the patient.

E100. The method of any one of E96-E99, wherein the corticosteroid is dexamethasone.

E101. The method of E100, wherein the dexamethasone is administered at a dose of 8 mg or 10 mg.

E102. The method of any one of E96-E101, wherein the subject is further premedicated with an antihistamine and an antipyretic at least 30 minutes prior to dosing with pivekimab sunirine anti-CD123 immunoconjugate.

E103. The method of E102, wherein the antihistamine is diphenhydramine and the antipyretic is acetaminophen or paracetamol.

E104. The method of E103, wherein diphenhydramine is administered intravenously at a dose of 25 mg to 50 mg; and (i) acetaminophen is administered orally or intravenously at a dose of 325 mg to 650 mg or (ii) paracetamol is administered orally or intravenously at a dose of 500 mg to 1000 mg.

E105. The method of any one of E1-E104, wherein pivekimab sunirine is administered intravenously at a dose of about 0.045 mg/kg once in a 21-day cycle.

E106. The method of E105, wherein the BPDCN is a frontline BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 5 months of the first administration, and wherein the duration of the CR, CRc, CRh, or CRi is at least 12 months.

E107. The method of E105, wherein the BPDCN is a R/R BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 2 months of the first administration, and wherein the duration of the CR, CRc, CRh, or CRi is at least 6 months or at least 7 months.

E108. The method of E105, wherein the BPDCN is a frontline BPDCN, and wherein the administration achieves a complete response (CR) or CR (clinical) with minimal residual skin abnormality (CRc), and wherein the duration of the CR or CRc is at least 9 months.

E109. The method of E105, wherein the BPDCN is a R/R BPDCN, and wherein the administration achieves a complete response (CR) or CR (clinical) with minimal residual skin abnormality (CRc), and wherein the duration of the CR or CRc is at least 9 months.

E110. The method of E105, wherein the BPDCN is a frontline BPDCN with no prior or concomitant hematologic malignancy (PCHM), and wherein the administration achieves a complete response (CR) or CR (clinical) with minimal residual skin abnormality (CRc), and wherein the duration of the CR or CRc is at least 9 months.

E111. Use of pivekimab sunirine in the method of any one of E1-E58 or in the preparation for a medicament for use in the method of any one of E1-E110.

E112. A method of preparing an adult human patient with blastic plasmacytoid dendritic cell neoplasm (BPDCN) for hematopoietic stem cell transplant (HSCT), the method comprising administering to the patient a safe and therapeutically effective dosing regimen of pivekimab sunirine anti-CD123 immunoconjugate, said immunoconjugate comprising a cleavable linker, an indolinobenzodiazepine pseudodimer payload, two heavy chains each having the amino acid sequence of SEQ ID NO:3, and two light chains each having the amino acid sequence of SEQ ID NO:4, wherein said dosing regimen comprises administering the immunoconjugate by infusion at a dose of about 0.045 mg/kg once in a 21-day cycle, wherein the patient is administered one or more cycles of infusion, wherein said safe and therapeutically effective dosing regimen results in the patient achieving a complete response (CR), CR (clinical) with minimal residual skin abnormality (CRc), CR (clinical) with partial hematological recovery (CRh), CR (clinical) with incomplete recovery (CRi), or a partial remission/partial response (PR), wherein said CR, CRc, CRh, CRi or PR comprises a hematologic recovery comprising a neutrophil count of greater than or equal to 500 neutrophils/microliter and a platelet count of greater than or equal to 50,000 platelets/microliter, wherein said dosing regimen results in a period of response or remission sufficient for said patient to be administered HSCT.

E113. The method of E112, wherein the corticosteroid is administered to a patient on the day prior and once, prior to and on the same day as administering an anti-CD123 immunoconjugate, e.g., pivekimab sunirine, to the patient.

E114. The method of E112, wherein the corticosteroid is administered to a patient twice on the day prior and once, prior to and on the same day as administering an anti-CD123 immunoconjugate, e.g., pivekimab sunirine, to the patient.

E115. The method of any one of E112-E114, wherein the corticosteroid is dexamethasone.

E116. The method of E115, wherein the dexamethasone is administered at a dose of 8 mg or 10 mg.

E117. The method of any one of E112-E116, wherein the subject is further premedicated with an antihistamine and an antipyretic at least 30 minutes prior to dosing with pivekimab sunirine anti-CD123 immunoconjugate.

E118. The method of E117, wherein the antihistamine is diphenhydramine and the antipyretic is acetaminophen or paracetamol.

E119. The method of E118, wherein diphenhydramine is administered intravenously at a dose of 25 mg to 50 mg and acetaminophen is administered orally or intravenously at a dose of 325 mg to 650 mg or paracetamol is administered orally or intravenously at a dose of 500 mg to 1000 mg.

E120. The method of any one of E112-E119, wherein the pivekimab sunirine is administered intravenously.

E121. The method of any one of E112-E119, wherein the pivekimab sunirine is administered by a controlled rate of infusion.

E122. The method of any one of E112-E119, wherein the pivekimab sunirine is administered in an infusion having a duration of no more than 30 minutes.

E123. The method of any one of E112-E119, wherein the pivekimab sunirine is administered in an infusion having a duration of about 15 to 30 minutes.

E124. The method of any one of E120-E123, wherein the controlled rate of infusion is from about 0.8 mL/min to about 1.7 mL/min.

E125. The method of any one of E120-E123, wherein the controlled rate of infusion is about 0.8 mL/min (about 50 mL/hour or about 0.165 mg/min) for the first 30 minutes.

E126. The method of any one of E120-E124, wherein the controlled rate of infusion is increased to about 1.7 mL/min (about 100 mL/hour or about 0.33 mg/min).

E127. The method of E112, wherein the subject has previously been treated with pivekimab sunirine.

E128. The method of E112, wherein the HSCT is administered about 4 to about 8 weeks after the administration of pivekimab sunirine.

E129. The method of E112, wherein the subject has a response with a duration of at least 6 months, at least 7 months, at least 8 months, at least 9 months, at least 10 months, at least 11 months, at least 12 months, at least 18 months, or at least 24 months.

E130. The method of E112, wherein the subject has received tagraxofusp prior to the administration.

E131. The method of E112, wherein the subject has previously been treated with venetoclax; a hypomethylating agent; cyclophosphamide, vincristine sulfate, and prednisone (CVP); and/or steroids.

E132. The method of E112, wherein the subject has previously been treated with cyclophosphamide, vincristine sulfate, doxorubicin hydrochloride, methotrexate, cytarabine, and dexamethasone (HyperCVAD); fludarabine, high-dose cytarabine, and G-CSF (FLAG); and/or cyclophosphamide, vincristine and doxorubicin (CHOP).

E133. The method of E112, wherein the BPDCN is relapsed or refractory (R/R) BPDCN.

E134. The method of E112, wherein the patient has received 1-3 prior systemic therapies.

E135. The method of E112, wherein the administration is a frontline therapy.

E136. The method of E112, wherein the BPDCN is de novo.

E137. The method of E112, wherein the subject has a prior or concomitant hematological malignancy (PCHM).

E138. The method of E112, wherein the subject has liver enzymes less than or equal to 2.5×the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

E139. The method of E112, wherein the BPDCN expresses multidrug resistance 1 (MDR1).

E140. The method of E112, wherein the BPDCN expresses P-glycoprotein (P-gp).

E141. The method of E112, wherein the pivekimab sunirine is administered intravenously.

### BRIEF DESCRIPTION OF THE FIGURES

FIG. 1 shows the interim Duration of Response (DOR) to treatment of frontline BPDCN with pivekimab sunirine. The median DOR was 12.7 months (95% CI 3.7-13.5). The median DOCR was 10.3 months (95% CI 3.7-12.9).

FIG. 2 shows the best percent decrease in bone marrow blasts following treatment of frontline BPDCN with pivekimab sunirine. In patients with bone marrow involvement at baseline, 100% (13/13) achieved bone marrow remission (<5% blasts). Of these, eleven out of thirteen achieved an overall response (composite CR+PR).

FIG. 3 shows the interim Duration of Response (DOR) to treatment of relapsed/refractory (R/R) BPDCN with pivekimab sunirine. The median DOR was 7.1 months (95% CI 1.8-NE). The median DOCR was 9.2 months (95% CI .8-NE).

FIG. 4 shows overall survival in total frontline BPDCN patients (N=30). The graph does not include data from three patients with frontline BPDCN with PCHM enrolled prior to Oct. 26, 2020. However, in the statistical outputs, the calculation for frontline BPDCN with PCHM (N=11) took into account data from one of the three pre-Oct. 26, 2020 patients.

FIG. 5 shows overall survival in R/R BPDCN patients (N=51).

FIG. 6 shows baseline and efficacy summaries from pivekimab sunirine and Elzonris monotherapy in BPDCN patients.

### DETAILED DESCRIPTION OF THE DISCLOSURE

### I. Definitions

To facilitate an understanding of the present disclosure, a number of terms and phrases are defined below.

The terms “IL-3Ra,” “Interleukine-3 Receptor alpha,” and “CD123,” as used interchangeably herein, refer to mammalian CD123 polypeptides, including, but not limited to, native CD123 polypeptides and isoforms of CD123 polypeptides, unless otherwise indicated. The terms encompass “full-length,” unprocessed CD123 polypeptides as well as any form of CD123 polypeptide that results from processing within the cell. The term also encompasses naturally occurring variants of CD123, e.g., those encoded by splice variants and allelic variants. The CD123 polypeptides described herein can be isolated from a variety of sources, such as from human tissue types or from another source, or prepared by recombinant or synthetic methods. Where specifically indicated, “CD123” can be used to refer to a nucleic acid that encodes a CD123 polypeptide. Human CD123 sequences are known and include, for example, those sequences associated with NCBI reference numbers NP_002174 & NM_002183 (protein and nucleic acid sequences for human CD123 variant 1), and NP_001254642 & NM_001267713 (protein and nucleic acid sequences for human CD123 variant 2).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “anti-CD123 antibody” or “an antibody that binds to CD123” refers to an antibody that is capable of binding CD123 with sufficient affinity such that the antibody is useful as a diagnostic and/or therapeutic agent in targeting CD123 (e.g., the antibody in pivekimab sunirine). The extent of binding of an anti-CD123 antibody to an unrelated, non-CD123 protein can be less than about 10% of the binding of the antibody to CD123 measured, e.g., by a radioimmunoassay (RIA).

The term “antibody fragment” refers to a portion of an intact antibody with a sufficient positive charge to bind to a cation exchange resin. An “antigen-binding fragment” refers to a portion of an intact antibody that binds to an antigen and has a sufficient positive charge to bind to a cation exchange resin. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “cysteine engineered” antibody or antigen-binding fragment thereof includes an antibody or antigen-binding fragment thereof with at least one cysteine (“Cys”) that is not normally present at a given residue of the antibody or antigen-binding fragment thereof light chain or heavy chain. Such Cys, which may also be referred to as “engineered Cys,” can be engineered using any conventional molecular biology or recombinant technology (e.g., by replacing the coding sequence for a non-Cys residue at the target residue with a coding sequence for Cys). For example, if the original residue is Ser with a coding sequence of 5′-UCU-3′, the coding sequence can be mutated (e.g., by site-directed mutagenesis) to 5′-UGU-3′, which encodes Cys. In certain aspects, the Cys engineered antibody or antigen-binding fragment thereof has an engineered Cys in the heavy chain. In certain aspects, the engineered Cys is in or near the CH3 domain of the heavy chain. In certain aspects, the engineered Cys is at residue 442 of the heavy chain (EU/OU numbering; EU index, Kabat et al, Sequences of Proteins of Immunological Interest, 5th Ed., NIH publication No. 91-3242, 1991, the entire contents of which are incorporated herein by reference). In certain aspects, the Fc region comprises a cysteine at one or more of positions 239, 282, 289, 297, 312, 324, 330, 335, 337, 339, 356, 359, 361, 383, 384, 398, 400, 440, 422, and 442, as numbered by the EU index. In certain aspects, any one or more of the following residues may be substituted with cysteine: V205 (Kabat numbering) of the light chain; A118 (EU numbering) of the heavy chain; and S400 (EU numbering) of the heavy chain Fc region. In certain aspects, the variable light chain domain, e.g., of an scFv, has a cysteine at Kabat position 100. In certain aspects, the variable heavy chain domain, e.g., of an scFv, has a cysteine at Kabat position 44. Cysteine engineered antibodies may be generated as described, e.g., in U.S. Pat. Nos. 7,521,541, 7,855,275, U.S. Published Application No. 2011/0033378 and WO 2011/005481.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′) 2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. (5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. (5th Ed., 1991, National Institutes of Health, Bethesda, Md.), “Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative aspects are described in the following.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain aspects, the polypeptides can occur as single chains or associated chains.

The term “immunoconjugate” or “conjugate” as used herein refers to a compound or a derivative thereof that is linked to a cell binding agent (i.e., an anti-CD123 antibody or fragment thereof) and is defined by a generic formula: C-A, wherein C=cytotoxin (e.g., such as an indolino-benzodiazepine (IGN) DNA-alkylator (e.g., DGN549-C)) and A=antibody or antigen-binding fragment thereof, e.g., an anti-CD123 antibody or antibody fragment. An immunoconjugate can optionally contain a linker and be defined by the generic formula C-L-A, wherein C=cytotoxin, L=linker, and A=antibody or antigen-binding fragment thereof, e.g., an anti-CD123 antibody or antibody fragment. Immunoconjugates can also be defined by the generic formula in reverse order: C-A or A-L-C. Immunoconjugates can also contain multiple cytotoxins (C) per antibody or antigen-binding fragment thereof (A) or multiple cytotoxins (C) and linkers (L) per antibody or antigen-binding fragment thereof (A).

A “linker” is any chemical moiety that is capable of linking a compound, usually a drug (such as IGN DNA-alkylators), to a cell-binding agent (such as an anti-CD123 antibody or a fragment thereof) in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art. In some aspects disclosed herein, the linker is a peptide linker.

The terms “cancer” and “cancerous” refer to or describe the physiological condition in mammals in which a population of cells are characterized by unregulated cell growth. Examples of cancer include blastic plasmacytoid dendritic cell neoplasm (BPDCN). The cancer can be a cancer that expresses CD123 (“CD123-expressing cancer”).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

A “refractory” cancer is one that progresses even though an anti-cancer treatment, such as a chemotherapy, is administered to the cancer patient. The cancer may be resistant at the beginning of treatment or it may become resistant during treatment.

A “primary refractory” cancer is one that does not achieve complete remission (CR) or complete remission with incomplete recovery (CRi) after a patient has received 2 cycles of intense chemotherapy.

A “relapsed” cancer is one in which the cancer or the signs and symptoms of a cancer returns after a period of improvement.

As it relates to cancer and cancer treatment, “remission” and “response” means a decrease, improvement or disappearance of one or more signs or symptoms of cancer.

Depending on context, the term “CR” means “Complete Remission” or “Complete Response”.

Depending on context, the term “PR” means “Partial Remission” or “Partial Response”.

The term “increased expression” or “overexpression” CD123 in a particular tumor, tissue, or cell sample refers to CD123 that is present at a level higher than that which is present in a healthy or non-diseased (native, wild type) tissue or cells of the same type or origin.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain aspects, a subject is successfully “treated” for cancer according to the methods of the present disclosure if the patient shows one or more of the following: a reduction in the number of cancer cells (e.g., BPDCN cells and CD123 positive cancer cells); a reduction in the tumor size or burden; a reduction in bone marrow infiltration; an inhibition (i.e., slowing to some extent and in a certain aspect, stopping) of cancer cell infiltration into peripheral organs (e.g., skin, CNS, lymph nodes and viscera); relief to some extent one or more of the symptoms associated with the cancer; and/or a favorable response such as complete remission (CR), complete remission with incomplete recovery (CRi), complete remission with partial hematologic recovery (CRh), CR without minimal residual disease (CRMRD-), complete remission clinical with minimal residual skin abnormality (CRc), partial remission (PR), and decrease in progressive disease (PD).

The term “therapeutically effective amount” refers to an amount of an antibody, immunoconjugate, or other drug effective to “treat” a disease or disorder in a subject or mammal. In the case of cancer, the therapeutically effective amount of the drug can reduce the number of cancer cells (e.g., BPDCN cells and CD123 positive cancer cells); reduce the tumor size or burden; reduce bone marrow infiltration; inhibit (i.e., slow to some extent and in a certain aspect, stop) cancer cell infiltration into peripheral organs (e.g., skin, CNS, lymph nodes and viscera); relieve to some extent one or more of the symptoms associated with the cancer; and/or result in a favorable response such as complete remission (CR), complete remission with incomplete recovery (CRi), complete remission with partial hematologic recovery (CRh), CR without minimal residual disease (CRMRD-), complete remission clinical with minimal residual skin abnormality (CRc), partial remission (PR), duration of response (DOR), and decrease in progressive disease (PD). In some embodiments, the therapeutically effective amount of the drug can increase the likelihood that the patient becomes eligible for stem cell transplantation. In some embodiments, the therapeutically effective amount of the drug has been shown to reduce the number of cancer cells (e.g., BPDCN cells and CD123 positive cancer cells); reduce the tumor size or burden; reduce bone marrow infiltration; inhibit (i.e., slow to some extent and in a certain aspect, stop) cancer cell infiltration into peripheral organs (e.g., skin, CNS, lymph nodes and viscera); relieve to some extent one or more of the symptoms associated with the cancer; and/or result in a favorable response such as complete remission (CR), complete remission with incomplete recovery (CRi), complete remission with partial hematologic recovery (CRh), CR without minimal residual disease (CRMRD-), complete remission clinical (CRc), partial remission (PR), duration of response (DOR), and decrease in progressive disease (PD).

The term “therapeutically safe amount” refers to an amount of an antibody, immunoconjugate (e.g., pivekimab sunirine), or other drug safe to “treat” a disease or disorder in a subject (e.g., human subject) or mammal. In some embodiments, the therapeutically safe amount of the drug (e.g., pivekimab sunirine) has been shown to cause no or only clinically manageable Infusion-related reactions (IRRs), no or only clinically manageable edema, and/or no or only clinically manageable veno-occlusive disease (VOD). In some embodiments, the therapeutically safe amount of the drug (e.g., pivekimab sunirine) has been shown to cause no capillary leak syndrome (CLS). In some embodiments, the therapeutically safe amount of the drug (e.g., pivekimab sunirine) has been shown to cause no or only clinically manageable Infusion-related reactions (IRRs), no or only clinically manageable edema, no or only clinically manageable veno-occlusive disease (VOD), and no capillary leak syndrome (CLS).

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50: 1S-10S (2009)). A favorable response can be assessed, for example, by complete remission (CR), complete remission with incomplete recovery (CRi); CR without minimal residual disease (CRMRD-); complete remission clinical (CRc); partial response (PR); a decrease in progressive disease (PD), or any combination thereof. The assessment criteria for evaluating the treatment of BPDCN are provided in Table 8 in Example 1 below.

As used herein the time to a “first response” is the time from the first administration of an anti-CD123 immunoconjugate, e.g., pivekimab sunirine, to the earliest of a complete response (CR), CR (clinical) with minimal residual skin abnormality (CRc), CR (clinical) with partial hematological recovery (CRh), CR (clinical) with incomplete recovery (CRi), or a partial response (PR).

The terms “clinical” and “clinical benefit” refer to an achievement by a patient of complete response or partial response for a time equal to or longer than 4 weeks. In some embodiments, the clinical benefit comprises a duration of response, i.e., CR, CRc, CRh, CRi, or PR that is at least 6 months, at least 7 months, at least 8 months, at least 9 months, at least 10 months, at least 11 months, at least 12 months, at least 18 months or at least 24 months.

The term “minimal residual skin abnormality” means: a) 100% clearance of all skin lesions from baseline with no new lesions in patients without lesions at baseline (CR); b) marked clearance of all skin lesions from baseline with residual hyperpigmentation or abnormality with BPDCN identified on biopsy (CRc and CRi); or, c) 50% to <100% clearance of all skin lesions from baseline with no new lesions in patients without lesions at baseline (PR).

The terms “line of treatment” or “line of therapy” refer to a therapeutic regimen that can include but is not limited to surgery, radiation therapy, chemotherapy, differentiating therapy, biotherapy, immune therapy, induction therapy, consolidation therapy, transplant, maintenance therapy, or the administration of one or more anti-cancer agents (e.g., a cytotoxic agent and/or an anti-proliferative compound).

The terms “first-line treatment,” “first-line therapy,” and “frontline therapy” refer to the preferred and standard initial treatment for a particular condition, e.g., induction therapy, consolidation therapy, transplant, maintenance therapy. As used herein, patients who receive “frontline therapy” are patients who have not received prior systemic therapy. However such a patient may have previously received a local therapy such as radiotherapy, surgical excision, and/or photodynamic therapy. These treatments differ from “second-line” therapies, which are tried when a first-line therapy does not work adequately. “Third-line” therapies are tried when a first-line therapy and a second-line therapy do not work adequately. “Salvage therapy” or “salvage regimen” is a treatment that is tried when the cancer has not responded to other treatments.

For example, a CD123 immunoconjugate (e.g., pivekimab sunirine) provided herein can be given as a first-line therapy, a second-line therapy, or a third-line therapy. A CD123 immunoconjugate (e.g., pivekimab sunirine) provided herein can be given as a line of therapy in patients having received at least 1, at least 2, or at least 3 lines of therapy (e.g., frontline therapy and one salvage therapy) prior to treatment with the CD123 immunoconjugate (e.g., pivekimab sunirine) provided herein. In some aspects, a CD123 immunoconjugate (e.g., pivekimab sunirine) can be given as a line of therapy in patients having received no more than 1, no more than 2, no more than 3, no more than 4, no more than 5, or no more than 6 lines of therapy.

The term “maintenance therapy” refers to therapy that is given to help keep cancer from coming back after it has disappeared following the initial therapy.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The term “premedication” refers to one or more additional agent(s) administered to a subject for a purpose that is not primarily or solely treating the primary disease (e.g., BPDCN) in the subject. For example, premedication may refer to one or more additional agent(s) used to prevent or ameliorate injection site side effects, infusion-related reactions, fever, and/or any other side effect associated with an anti-CD123 antibody. In embodiments, a premedication is administered using a premedication regimen.

The term “controlled rate of infusion” means administering a therapeutic agent in a manner that minimizes the variability of the average amount of the therapeutic agent, (e.g., pivekimab sunirine) that is administered to the patient over a period of time to, e.g., ensure a constant delivery of the therapeutic agent over a prolonged time. “Controlled rate of infusion” does not mean administration via IV push.

The term “subject” or “patient” refers to a human who is to be the recipient of a particular treatment.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). It is understood that wherever aspects are described herein with the language “about” a numeric value or range, otherwise analogous aspects referring to the specific numeric value or range (without “about”) are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### II. Anti-CD123 Immunoconjugates

Described herein are methods of administering immunoconjugates that specifically bind CD123. Immunoconjugates that specifically bind to CD123 are referred to herein as “CD123-immunoconjugates” or “anti-CD123 immunoconjugates.” Such immunoconjugates comprise an anti-CD123 antibody or antigen-binding fragment thereof and a drug (e.g., a cytotoxic agent). The drug (e.g., a cytotoxic agent) can be attached to the anti-CD123 antibody or antigen-binding fragment thereof by a linker.

In some aspects, the anti-CD123 immunoconjugate is pivekimab sunirine.

In some aspects, the anti-CD123 antibodies or antigen-binding fragments thereof are humanized antibodies or antigen-binding fragments thereof. In some aspects, the humanized antibody or fragment is a resurfaced antibody or antigen-binding fragment thereof. In other aspects, the antibodies or antigen-binding fragments thereof is a fully human antibody or antigen-binding fragment thereof.

In certain aspects, the immunoconjugate is represented by the following formula:

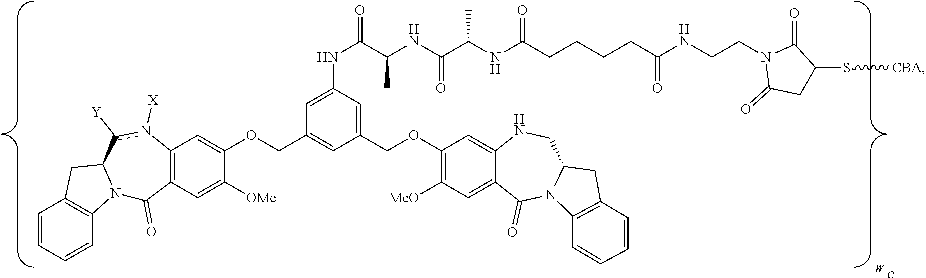

or a pharmaceutically acceptable salt thereof, wherein the double line [symbol] between N and C represents a single bond or a double bond, provided that when it is a double bond, X is absent and Y is —H, and when it is a single bond, X is —H, and Y is —OH or —SO3M.

By way of example, an anti-CD123 antibody or antigen-binding fragment thereof can be in an immunoconjugate used in the present methods. Anti-CD123 antibodies or antigen-binding fragments thereof have been described (see e.g., U.S. Pat. No. 10,077,313 B2, the contents of which are herein incorporated by reference in their entirety). The anti-CD123 antibody or antigen-binding fragment thereof can be the huCD123-6Gv4.7 (“G4723A”) antibody (see WO 2017/004025, WO 2017/004026, and WO 2019/060718, the contents of each of which are herein incorporated by reference in their entireties) or can contains sequences of the G4723A antibody, e.g., as shown below in Tables 1-3. For example, an anti-CD123 antibody or antigen-binding fragment thereof for use in the methods provided herein can comprise variable heavy chain CDR-1, CDR-2, and CDR-3 comprising the sequences of SEQ ID NOs: 5, 6, and 7, respectively and/or variable light chain CDR-1, CDR-2, and CDR-3 comprising the sequences of SEQ ID NOs: 8, 9, and 10, respectively. An anti-CD123 antibody or antigen-binding fragment thereof for use in the methods provided herein can comprise a variable heavy chain domain comprising the sequence set forth in SEQ ID NO:1. An anti-CD123 antibody or antigen-binding fragment thereof for use in the methods provided herein can comprise a variable light chain domain comprising the sequence set forth in SEQ ID NO:2. An anti-CD123 antibody or antigen-binding fragment thereof for use in the methods provided herein can comprise a variable heavy chain domain comprising the sequence set forth in SEQ ID NO:1 and a variable light chain domain comprising the sequence set forth in SEQ ID NO:2. An anti-CD123 antibody or antigen-binding fragment thereof for use in the methods provided herein can comprise a heavy chain comprising the sequence set forth in SEQ ID NO:3. An anti-CD123 antibody or antigen-binding fragment thereof for use in the methods provided herein can comprise a light chain comprising the sequence set forth in SEQ ID NO:4. An anti-CD123 antibody or antigen-binding fragment thereof for use in the methods provided herein can comprise a heavy chain comprising the sequence set forth in SEQ ID NO:3 and a light chain comprising the sequence set forth in SEQ ID NO:4.

TABLE 1
huCD123-6Gv4.7 Heavy and Light
Chain Variable Regions

| Name | Sequence |
|---|---|
| huCD123-6Gv7 | QVQLVQSGAEVKKPGASVKVSCKASGYIFTSSIMH |
| Heavy Chain | WVRQAPGQGLEWIGYIKPYNDGTKYNEKFKGRATL |
| Variable | TSDRSTSTAYMELSSLRSEDTAVYYCAREGGNDYY |
| Region | DTMDYWGQGTLVTVSS (SEQ ID NO: 1) |
| huCD123-6Gv4 | DIQMTQSPSSLSASVGDRVTITCRASQDINSYLSW |
| Light Chain | FQQKPGKAPKTLIYRVNRLVDGVPSRFSGSGSGND |
| Variable | YTLTISSLQPEDFATYYCLQYDAFPYTFGQGTKVE |
| Region | IKR (SEQ ID NO: 2) |

TABLE 2
huCD123-6Gv4.7-C442 Full Length
Heavy and Light Chain

| Name | Sequence |
|---|---|
| huCD123- | QVQLVQSGAEVKKPGASVKVSCKASGYIFTSSIMHWVR |
| 6Gv7- | QAPGQGLEWIGYIKPYNDGTKYNEKFKGRATLTSDRST |
| C442 | STAYMELSSLRSEDTAVYYCAREGGNDYYDTMDYWGQG |
| Full | TLVTVSSASTKGPSVFPLAPSSKSTSGGTAALGCLVKD |
| Length | YFPEPVTVSWNSGALTSGVHTFPAVLQSSGLYSLSSVV |
| Heavy | TVPSSSLGTQTYICNVNHKPSNTKVDKKVEPKSCDKTH |
| Chain | TCPPCPAPELLGGPSVFLFPPKPKDTLMISRTPEVTCV |
| | VVDVSHEDPEVKFNWYVDGVEVHNAKTKPREEQYNSTY |
| | RVVSVLTVLHQDWLNGKEYKCKVSNKALPAPIEKTISK |
| | AKGQPREPQVYTLPPSRDELTKNQVSLTCLVKGFYPSD |
| | IAVEWESNGQPENNYKTTPPVLDSDGSFFLYSKLTVDK |
| | SRWQQGNVFSCSVMHEALHNHYTQKSLCLSPG (SEQ |
| | ID NO: 3) |
| huCD123- | DIQMTQSPSSLSASVGDRVTITCRASQDINSYLSWFQQ |
| 6Gv4 | KPGKAPKTLIYRVNRLVDGVPSRFSGSGSGNDYTLTIS |
| Full | SLQPEDFATYYCLQYDAFPYTFGQGTKVEIKRTVAAPS |
| Length | VFIFPPSDEQLKSGTASVVCLLNNFYPREAKVQWKVDN |
| Light | ALQSGNSQESVTEQDSKDSTYSLSSTLTLSKADYEKHK |
| Chain | VYACEVTHQGLSSPVTKSFNRGEC (SEQ ID |
| | NO: 4) |

TABLE 3
huCD123-6Gv4.7 Variable Heavy and
Light Chain Complementary
Determining Regions

| Name | Sequence |
|---|---|
| huCD123-6Gv7 Variable | SSIMH |
| Heavy Chain CDR1 | (SEQ ID NO: 5) |
| huCD123-6Gv7 Variable | YIKPYNDGTKYNEKFKG |
| Heavy Chain CDR2 | (SEQ ID NO: 6) |
| huCD123-6Gv7 Variable | EGGNDYYDTMDY |
| Heavy Chain CDR3 | (SEQ ID NO: 7) |
| huCD123-6Gv4 Variable | RASQDINSYLS |
| Light Chain CDR1 | (SEQ ID NO: 8) |
| huCD123-6Gv4 Variable | RVNRLVD |
| Light Chain CDR2 | (SEQ ID NO: 9) |
| huCD123-6Gv4 Variable | LQYDAFPYT |
| Light Chain CDR3 | (SEQ ID NO: 10) |

An anti-CD123 antibody or antigen-binding fragment thereof for use in the methods provided herein can bind to an epitope within amino acids 205 to 346 of human CD123.

An anti-CD123 antibody or antigen-binding fragment thereof for use in methods provided herein can be recombinantly produced. For example, an anti-CD123 antibody or antigen-binding fragment thereof for use in the methods provided herein can be produced in a mammalian cell line, e.g., a CHO cell.

An anti-CD123 antibody or antigen-binding fragment thereof for use in the methods provided herein can be a cysteine-engineered antibody or fragment. Cysteine-engineered antibodies can be covalently conjugated to cytotoxic agents of interest to generate immunoconjugates.

As used herein, the expression “linked to a cell-binding agent” or “linked to an anti-CD123 antibody or fragment” refers to a conjugate molecule comprising at least one cytotoxic agent bound to a cell-binding agent, e.g., anti-CD123 antibody or fragment, via a suitable linking group, or a precursor thereof. Linkers include, for example, peptide linkers.

An immunoconjugate can contain multiple cytotoxic agents bound to an antibody or antigen-binding fragment thereof. As provided herein, in certain instances, about 1 to about 3 drug molecules e.g., cytotoxic agents, are linked to an anti-CD123 antibody or antigen-binding fragment thereof. In one aspect, an immunoconjugate comprises 1, 2, or 3, cytotoxic agents per antibody or antigen-binding fragment thereof.

A composition comprising immunoconjugates can contain immunoconjugates with varying numbers of cytotoxic agents bound per antibody or antigen-binding fragment thereof. Thus, compositions comprising immunoconjugates can contain an average number of cytotoxic agents bound per antibody or antigen-binding fragment thereof. In one aspect, a pharmaceutical composition comprising anti-CD123 immunoconjugates comprises about 1 to about 3 cytotoxic agents per anti-CD123 antibody or antigen-binding fragment thereof, about 1.5 to about 2.5 cytotoxic agents per anti-CD123 antibody or antigen-binding fragment thereof, about 1.5 to about 2.1 cytotoxic agents per anti-CD123 antibody or antigen-binding fragment thereof, or about 1.5 to about 2.0 cytotoxic agents cytotoxic agents per anti-CD123 antibody or antigen-binding fragment thereof.

In certain instances, a pharmaceutical composition for use in the methods provided herein comprises anti-CD123 immunoconjugates comprising about 1 to about 3 cytotoxic agents per antibody or antigen-binding fragment thereof, for example, wherein the average number of cytotoxic agents per antibody or antigen-binding fragment thereof is from about 1 to about 3 (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0).

In certain instances, a pharmaceutical composition for use in the methods provided herein comprises anti-CD123 immunoconjugates with an average of about 1±0.2, about 1.1±0.2, about 1.2±0.2, about 1.3±0.2, about 1.4±0.2, about 1.5±0.2, about 1.6±0.2, about 1.7±0.2, about 1.8±0.2, about 1.9±0.2, about 2.0±0.2, about 2.1±0.2, 2.2±0.2, 2.3±0.2, 2.4±0.2, 2.5±0.2, or 2.6±0.2 drug molecules (e.g., cytotoxic agents) attached per antibody or antigen-binding fragment thereof. In certain aspects, a pharmaceutical composition provided herein comprises anti-CD123 immunoconjugates with an average of about 1.5 to 2.1 drug molecules (e.g., cytotoxic agents) per antibody.

The antibodies or antigen-binding fragments thereof for use in the present disclosure may be linked to cytotoxic agents, for example, through linkage with the Lys side chain amino group, the Cys side chain thiol group, or an oxidized N-terminal Ser/Thr. Cytotoxic agents include, for example, DNA alkylating agents such as indolino-benzodiazepene (IGN) DNA alkylators. In certain instances, a cytotoxic agent is an indolino-benzodiazepine pseudodimer. In certain instances, an anti-CD123 immunoconjugate for use in the present disclosure comprises DGN549-C.

### III. Methods of Treatment and Use

As provided herein, anti-CD123 immunoconjugates such as pivekimab sunirine can be used to treat BPDCN in a subject. In some aspects, the methods comprise administering a safe and effective amount of the anti-CD123 immunoconjugate, e.g., pivekimab sunirine to the subject. In some aspects, the method does not comprise administering a hematopoietic stem cell transplant (HSCT) to the subject to the subject.

As provided herein, anti-CD123 immunoconjugates such as pivekimab sunirine can also be used to prepare a subject with BPDCN for HSCT. In some aspects, provided herein are methods of treating BPDCN in a subject by administering a safe and effective amount of anti-CD123 immunoconjugate to the subject and then an HSCT to the subject. In some aspects, provided herein are methods of treating BPDCN in a subject by administering a HSCT to the subject, wherein the subject was previously treated with an anti-CD123 immunoconjugate such as pivekimab sunirine. The HSCT can be allogeneic. Alternatively, the HSCT can be autologous.

In some aspects, an anti-CD123 immunoconjugate, e.g., pivekimab sunirine, can be administered as a maintenance dose.

The risks of infusion-related reactions may be heightened if patients are administered pivekimab sunirine by IV push or via an infusion rate that is not a controlled rate of infusion. In some aspects, the anti-CD123 immunoconjugate, e.g., pivekimab sunirine, is administered intravenously, e.g., in an infusion having a duration of no more than 30 minutes. In aspects, the pivekimab sunirine is administered via an infusion having a duration of about 15 to about 30 minutes. In aspects, the pivekimab sunirine is administered by a controlled rate of infusion. In aspects, the pivekimab sunirine is administered by a controlled rate of infusion, where the infusion rate is from about 0.8 mL/min to about 1.7 mL/min. In some aspects, the pivekimab sunirine is administered at an infusion rate of about 0.8 mL/min (about 50 mL/hour or about 0.165 mg/min) for the first 30 minutes. In aspects, the infusion rate may be increased to about 1.7 mL/min (about 100 mL/hour or about 0.33 mg/min). In aspects, the infusion rate may be increased to about 1.7 mL/min (about 100 mL/hour or about 0.33 mg/min), if well tolerated whereby no infusion-related reactions are observed in the patient. In aspects, the pivekimab sunirine is not administered by IV push. In embodiments, administering the pivekimab sunirine by a controlled rate of infusion, where the infusion rate is from about 0.8 mL/min to about 1.7 mL/min may prevent the patient from experiencing infusion-related reactions or minimize the effects of infusion-related reactions. In aspects, the pivekimab sunirine is administered via an infusion having a duration of 15 to 30 minutes. In aspects, the pivekimab sunirine is administered by a controlled rate of infusion. In aspects, the pivekimab sunirine is administered by a controlled rate of infusion, where the infusion rate is from 0.8 mL/min to 1.7 mL/min. In some aspects, the pivekimab sunirine is administered at an infusion rate of 0.8 mL/min (50 mL/hour or 0.165 mg/min) for the first 30 minutes. In aspects, the infusion rate may be increased to 1.7 mL/min (100 mL/hour or 0.33 mg/min). In aspects, the infusion rate may be increased to 1.7 mL/min (100 mL/hour or 0.33 mg/min), if well tolerated whereby no infusion-related reactions are observed in the patient. In aspects, the pivekimab sunirine is not administered by IV push. In embodiments, administering the pivekimab sunirine by a controlled rate of infusion, where the infusion rate is from 0.8 mL/min to 1.7 mL/min may prevent the patient from experiencing infusion-related reactions or minimize the effects of infusion-related reactions.

### III (A). Cancer Selection

Cancers that can be treated by the methods provided herein include BPDCN. In certain aspects, the BPDCN is a CD123-expressing BPDCN. The BPDCN can be a frontline BPDCN. The BPDCN can also be a relapsed or refractory (R/R) BPDCN. The BPDCN can be in a patient who is not a candidate for stem cell transplant.

In certain aspects, the BPDCN is a frontline BPDCN. In some aspects, the BPDCN is de novo. In some aspects, the subject has a prior or concomitant hematological malignancy (PCHM). In some aspects, the PCHM does not require immediate therapy. In some aspects, the PCHM is in remission and the subject has completed all therapies for the PCHM at least 6 months prior to the administration of the anti-CD123 immunoconjugate, e.g., pivekimab sunirine.

In certain aspects, the BPDCN is a relapsed BPDCN. In certain aspects, the BPDCN is a refractory BPDCN.

In some aspects, the subject with BPDCN has involvement of skin, bone marrow, lymph node, viscera, or a combination thereof in the BPDCN. In some aspects, the subject with BPDCN has involvement of skin, bone marrow, lymph node, or a combination thereof in the BPDCN.

In some aspects, the subject has a history of central nervous system (CNS) involvement in the BPDCN. In some aspects, the CNS involvement was treated locally and the subject has had at least one lumbar puncture with no evidence of CNS disease prior to the administration of the anti-CD123 immunoconjugate, e.g., pivekimab sunirine.

In certain aspects, the BPDCN expresses multidrug resistance 1 (MDR1). In certain aspects, the BPDCN expresses the multidrug resistance (MDR)-related P-glycoprotein (P-gp). In certain aspects, the BPDCN overexpresses MDR1 and P-gp.

In certain aspects, the BPDCN or the subject with the BPDCN has an FLT3-ITD mutation. In certain aspects, the BPDCN or the subject with the BPDCN does not have an FLT3-ITD mutation.

In certain aspects, the BPDCN is present in the subject as minimal residual disease (MRD). The methods provided herein can covert MRD+ patients to MRD-patients. The methods provided herein can also increase the relapse-free survival time (e.g., the median relapse-free survival time) in MRD+ patients.

In some aspects, at least about 80% of cells of the BPDCN are CD123+ (e.g., as determined by local flow cytometry or immunohistochemistry).

In some aspects, it has been determined prior to the administration of the anti-CD123 immunoconjugate (e.g., pivekimab sunirine) that at least 80% of cells of the BPDCN are CD123-positive (e.g., as determined by local flow cytometry or immunohistochemistry).

In some aspects, the BPDCN has not previously been treated. In some aspects, the BPDCN has not previously been treated with a systemic therapy.

In some aspects, the subject has received one prior treatment regimen (e.g., one prior systemic treatment regimen) for the BPDCN. In some aspects, the subject has received at least one prior treatment regimen (e.g., at least one prior systemic treatment regimen) for the BPDCN. In some aspects, the subject has received two prior treatment regimens (e.g., two prior systemic treatment regimens) for the BPDCN. In some aspects, the subject has received at least two prior treatment regimens (e.g., at least two prior systemic treatment regimens) for the BPDCN. In some aspects, the subject has received at least three prior treatment regimens (e.g., three prior systemic treatment regimens) for the BPDCN. In some aspects, the subject has received three prior treatment regimens (e.g., at least three prior systemic treatment regimens) for the BPDCN. In some aspects, the subject has received one to three prior treatment regimens (e.g. at one to three prior systemic treatment regimens) for the BPDCN. In some aspects, the subject has received no more than six prior treatment regimens (e.g., no more than six prior systemic treatment regiments) for the BPDCN. In certain aspects, the subject has received at least one prior treatment, but no more than six prior treatment regimens (e.g., at least one, but no more than six, prior systemic treatment regimens) for the BPDCN.

In some aspects, the BPDCN has previously been treated with venetoclax; a hypomethylating agent; cyclophosphamide, vincristine sulfate, and prednisone (CVP); and/or steroids. In some aspects, the BPDCN has previously been treated with cyclophosphamide, vincristine sulfate, doxorubicin hydrochloride, methotrexate, cytarabine, and dexamethasone (HyperCVAD); fludarabine, high-dose cytarabine, and G-CSF (FLAG) and/or cyclophosphamide, vincristine and doxorubicin (CHOP).

In one aspect the BPDCN has previously been treated with a BCL-2 inhibitor. In one aspect, the BPDCN has not previously been treated with a BCL-2 inhibitor (i.e., the patient is “BCL-2 inhibitor naive”).

In one aspect the BPDCN has previously been treated with venetoclax. In one aspect, the BPDCN has not previously been treated with venetoclax (i.e., the patient is “venetoclax naive”).

In one aspect the BPDCN has previously been treated with a hypomethylating agent. In one aspect, the BPDCN has not previously been treated with a hypomethylating agent (i.e., the patient is “hypomethylating agent naive”).

In one aspect the BPDCN has previously been treated with azacitidine. In one aspect, the BPDCN has not previously been treated with azacitidine (i.e., the patient is “azacitidine naive”).

In one aspect the BPDCN has previously been treated with decitabine. In one aspect, the BPDCN has not previously been treated with decitabine (i.e., the patient is “decitabine naive”).

In one aspect the BPDCN has previously been treated with tagraxofusp. In one aspect, the BPDCN has not previously been treated with tagraxofusp (i.e., the patient is “tagraxofusp naive”).

In one aspect, the patient had previously received a stem cell transplant (e.g., an allogeneic stem cell transplant or an autologous stem cell transplant) prior to the administration of the anti-CD123 immunoconjugate. In some aspects, the stem cell transplant was received at least 120 days prior to the administration of the anti-CD123 immunoconjugate. In another aspect, the patient has not previously received a stem cell transplant prior to the administration of the anti-CD123 immunoconjugate, e.g., pivekimab sunirine.

In one aspect, the subject received a local therapy, e.g., radiotherapy, prior to the administration of the anti-CD123 immunoconjugate, e.g., pivekimab sunirine. In one aspect, the local therapy, e.g., radiotherapy, was administered at least 14 days prior to the administration of the anti-CD123 immunoconjugate, e.g., pivekimab sunirine

In one aspect, the subject has not received immunosuppressive therapy for at least 14 days and/or has not received a systemic anti-cancer therapy for at least 14 days prior to the administration prior to the administration of the anti-CD123 immunoconjugate, e.g., pivekimab sunirine.

In one aspect, the subject to receive the anti-CD123 immunoconjugate, e.g., pivekimab sunirine, has liver enzymes less than or equal to 2.5×the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

### III (B). Dosing

As provided herein, an anti-CD123 immunoconjugate (e.g., pivekimab sunirine) can be administered at a particular dose and/or at particular timing intervals. Administration of anti-CD123 immunoconjugates (e.g., pivekimab sunirine) can be, for example, intravenous. In certain aspects, the anti-CD123 immunoconjugate (e.g., pivekimab sunirine) is administered once in a three-week (21-day) cycle. In certain aspects, one cycle of treatment is therapeutically effective. In certain aspects, two cycles of treatment are therapeutically effective. In certain aspects, three cycles of treatment are therapeutically effective. In certain aspects, one to three cycles of treatment are therapeutically effective. In certain aspects, one to four cycles of treatment are therapeutically effective. In certain aspects, one to twelve cycles of treatment are therapeutically effective.

In some aspects, patients can be treated for one cycle (e.g., a 21-day cycle), e.g., wherein the anti-CD123 immunoconjugate (e.g., pivekimab sunirine) is administered once in the cycle. In some aspects, patients can be treated for at least two cycles (e.g., 21-day cycles), e.g., wherein the anti-CD123 immunoconjugate (e.g., pivekimab sunirine) is administered once per cycle. In some aspects, patients can be treated for at least three cycles (e.g., 21-day cycles), e.g., wherein the anti-CD123 immunoconjugate (e.g., pivekimab sunirine) is administered once per cycle. In some aspects, patients can be treated for at least four cycles (e.g., 21-day cycles), e.g., wherein the anti-CD123 immunoconjugate (e.g., pivekimab sunirine) is administered once per cycle. In some aspects, patients can be treated for at least five cycles (e.g., 21-day cycles), e.g., wherein the anti-CD123 immunoconjugate (e.g., pivekimab sunirine) is administered once per cycle. In some aspects, patients can be treated for at least six cycles (e.g., 21-day cycles), e.g., wherein the anti-CD123 immunoconjugate (e.g., pivekimab sunirine) is administered once per cycle. In some aspects, patients can be treated for at least seven cycles (e.g., 21-day cycles), e.g., wherein the anti-CD123 immunoconjugate (e.g., pivekimab sunirine) is administered once per cycle. In some aspects, patients can be treated for at least eight cycles (e.g., 21-day cycles), e.g., wherein the anti-CD123 immunoconjugate (e.g., pivekimab sunirine) is administered once per cycle. In some aspects, patients can be treated for at least nine cycles (e.g., 21-day cycles), e.g., wherein the anti-CD123 immunoconjugate (e.g., pivekimab sunirine) is administered once per cycle. In some aspects, patients can be treated for at least ten cycles (e.g., 21-day cycles), e.g., wherein the anti-CD123 immunoconjugate (e.g., pivekimab sunirine) is administered once per cycle. In some aspects, patients can be treated for at least eleven cycles (e.g., 21-day cycles), e.g., wherein the anti-CD123 immunoconjugate (e.g., pivekimab sunirine) is administered once per cycle. In some aspects, patients can be treated for at least twelve cycles (e.g., 21-day cycles), e.g., wherein the anti-CD123 immunoconjugate (e.g., pivekimab sunirine) is administered once per cycle.

In some aspects, patients can be treated for one to ten cycles (e.g., 21-day cycles), e.g., wherein the anti-CD123 immunoconjugate (e.g., pivekimab sunirine) is administered once per cycle. In some aspects, patients can be treated for two to ten cycles (e.g., 21-day cycles), e.g., wherein the anti-CD123 immunoconjugate (e.g., pivekimab sunirine) is administered once per cycle. In some aspects, patients can be treated for three to ten cycles (e.g., 21-day cycles), e.g., wherein the anti-CD123 immunoconjugate (e.g., pivekimab sunirine) is administered once per cycle. In some aspects, patients can be treated for four to ten cycles (e.g., 21-day cycles), e.g., wherein the anti-CD123 immunoconjugate (e.g., pivekimab sunirine) is administered once per cycle. In some aspects, patients can be treated for five to ten cycles (e.g., 21-day cycles), e.g., wherein the anti-CD123 immunoconjugate (e.g., pivekimab sunirine) is administered once per cycle.

In some aspects, patients can be treated for one to twelve cycles (e.g., 21-day cycles), e.g., wherein the anti-CD123 immunoconjugate (e.g., pivekimab sunirine) is administered once per cycle. In some aspects, patients can be treated for two to twelve cycles (e.g., 21-day cycles), e.g., wherein the anti-CD123 immunoconjugate (e.g., pivekimab sunirine) is administered once per cycle. In some aspects, patients can be treated for three to twelve cycles (e.g., 21-day cycles), e.g., wherein the anti-CD123 immunoconjugate (e.g., pivekimab sunirine) is administered once per cycle. In some aspects, patients can be treated for four to twelve cycles (e.g., 21-day cycles), e.g., wherein the anti-CD123 immunoconjugate (e.g., pivekimab sunirine) is administered once per cycle. In some aspects, patients can be treated for five to twelve cycles (e.g., 21-day cycles), e.g., wherein the anti-CD123 immunoconjugate (e.g., pivekimab sunirine) is administered once per cycle.

In certain aspects, about 0.015 mg/kg to about 0.045 mg/kg of an anti-CD123 immunoconjugate (e.g., pivekimab sunirine) is administered, e.g., once in a three-week (21-day) cycle. In some aspects, about 0.015 mg/kg of an anti-CD123 immunoconjugate (e.g., pivekimab sunirine) is administered, e.g., once in a three-week (21-day) cycle. In some aspects, about 0.045 mg/kg of an anti-CD123 immunoconjugate (e.g., pivekimab sunirine) is administered, e.g., once in a three-week (21-day) cycle.

In some aspects, a method provided herein further comprises administering a reduced dose of the anti-CD123 immunoconjugate (e.g., pivekimab sunirine) after a dose-limiting toxicity has occurred in the subject and has been reduced to baseline or ≤Grade 2.

### III (C). Clinical Outcomes Achieved Using Anti-CD123 Immunoconjugates

As provided herein, administration of an anti-CD123 immunoconjugate such as pivekimab sunirine can achieve desirable clinical outcomes in patients who are candidates for stem cell transplant as well as in patients who are not candidates for stem cell transplant. In a particular aspect, administration of an anti-CD123 immunoconjugate such as pivekimab sunirine achieves a clinical benefit in a patient. In a particular aspect, administration of an anti-CD123 immunoconjugate such as pivekimab sunirine achieves a clinical benefit in a patient without significantly increasing the risk of capillary leak syndrome (CLS). In an additional particular aspect, administration of an anti-CD123 immunoconjugate such as pivekimab sunirine achieves a clinical benefit without CLS.

As demonstrated herein, administration of an anti-CD123 immunoconjugate such as pivekimab sunirine can achieve bone marrow remission in a subject with BPDCN with bone marrow involvement. As demonstrated herein, administration of an anti-CD123 immunoconjugate such as pivekimab sunirine can achieve bone marrow remission in a subject with BPDCN with bone marrow involvement without significantly increasing the risk of capillary leak syndrome (CLS). As demonstrated herein, administration of an anti-CD123 immunoconjugate such as pivekimab sunirine can achieve bone marrow remission in a subject with BPDCN with bone marrow involvement CLS.

As also demonstrated herein, administration of an anti-CD123 immunoconjugate such as pivekimab sunirine can produce a complete response (CR), CR (clinical) with minimal residual skin abnormality (CRc), CR (clinical) with partial hematological recovery (CRh), CR (clinical) with incomplete recovery (CRi), or a partial response (PR) in a subject with BPDCN. In some aspects, administration of an anti-CD123 immunoconjugate such as pivekimab sunirine can produce a complete response (CR) or CR (clinical) with minimal residual skin abnormality (CRc) in a subject with BPDCN. In some aspects, the time to the first response is about 4 months or less. In some aspects, the time to the first response is about 3.5 months or less. In some aspects, the time to the first response is about 2 months or less. In some aspects, the time to the first response is about 1.5 months or less. In some aspects, the response is produced without significantly increasing the risk of capillary leak syndrome (CLS). In some aspects, the response is produced with no CLS.

In some aspects, the time to the first response is about 0.5 to about 4 months. In some aspects, the time to the first response is about 0.5 to about 3.5 months. In some aspects, the time to the first response is about 0.5 to about 2 months. In some aspects, the time to the first response is about 0.5 to about 1.5 months.

In some aspects, the time to the first response is about 1 to about 4 months. In some aspects, the time to the first response is about 1 to about 3.5 months. In some aspects, the time to the first response is about 1 to about 2 months. In some aspects, the time to the first response is about 1 to about 1.5 months.

In some aspects, the duration of the response, i.e., the CR, CRc, CRh, CRi, or PR is at least 6 months, at least 7 months, at least 8 months, at least 9 months, at least 10 months, at least 11 months or at least 12 months. In some aspects, the duration of the response, i.e., the CR, CRc, CRh, CRi, or PR is about 6 months to about 2 years.

In some aspects, the duration of the CR or CRc is at least 6 months, at least 7 months, at least 8 months, at least 9 months, at least 10 months, at least 11 months or at least 12 months. In some aspects, the duration of the CR or CRc is about 6 months to about 2 years.

In some aspects, the time to the first response in a patient with frontline BPDCN is about 0.5 to about 4 months. In some aspects, the duration of a response in a patient with frontline BPDCN is at least 1 year. In some aspects, the duration of the response, i.e., the CR, CRc, CRh, CRi, or PR in a patient with frontline BPDCN is at least 8 months, at least 9 months, at least 10 months, at least 11 months or at least 12 months. In some aspects, the duration of the CR or CRc in a patient with frontline BPDCN is at least 8 months, at least 9 months, at least 10 months, at least 11 months or at least 12 months. In some aspects, the duration of a response in a patient with frontline BPDCN is about 6 months to about 2 years. In some aspects, the duration of a response in a patient with frontline BPDCN is about 8 months to about 2 years. In some aspects, the duration of a response in a patient with frontline BPDCN is about 9 months to about 2 years. In some aspects, the duration of a response in a patient with frontline BPDCN is about 10 months to about 2 years. In some aspects, the response is produced without significantly increasing the risk of capillary leak syndrome (CLS). In some aspects, the response is produced without CLS. In some aspects, the subject has frontline BPDCN with no prior or concomitant hematological malignancy (PCHM).

In some aspects, the time to the first response in a patient with R/R BPDCN is about 0.5 to about 4 months. In some aspects, the duration of a response in a patient with R/R BPDCN is at least 6 months. In some aspects, the duration of the response, i.e., the CR, CRc, CRh, CRi, or PR in a patient with R/R BPDCN is at least 8 months, at least 9 months, at least 10 months, at least 11 months or at least 12 months. In some aspects, the duration of the CR or CRc in a patient with R/R BPDCN is at least 8 months, at least 9 months, at least 10 months, at least 11 months or at least 12 months. In some aspects, the duration of a response in a patient with R/R BPDCN is about 6 months to about 1 year. In some aspects, the duration of a response in a patient with R/R BPDCN is about 8 months to about 2 years. In some aspects, the duration of a response in a patient with R/R BPDCN is about 9 months to about 2 years. In some aspects, the duration of a response in a patient with frontline BPDCN is about 10 months to about 2 years. In some aspects, the response is produced without significantly increasing the risk of capillary leak syndrome (CLS). In some aspects, the response is produced without CLS. In some aspects, the subject has frontline BPDCN with no prior or concomitant hematological malignancy (PCHM).

In some aspects, administration of an anti-CD123 immunoconjugate such as pivekimab sunirine produces a CR, CRc, CRh, or CRi, and the time to the CR, CRc, CRh, or CRi is about 5 months or less. In some aspects, the time to the complete response is about 4 months or less. In some aspects, the time to the CR, CRc, CRh, or CRi is about 3 months or less. In some aspects, the time to the CR, CRc, CRh, or CRi is about 2 months or less. In some aspects, the time to the CR, CRc, CRh, or CRi is about 1.5 months or less. In some aspects, the response is produced without significantly increasing the risk of capillary leak syndrome (CLS). In some aspects, the response is produced without CLS.

In some aspects, administration of an anti-CD123 immunoconjugate such as pivekimab sunirine produces a CR or CRc, and the time to the CR or CRc is about 5 months or less. In some aspects, the time to the complete response is about 4 months or less. In some aspects, the time to the CR or CRc is about 3 months or less. In some aspects, the time to the CR or CRc is about 2 months or less. In some aspects, the time to the CR or CRc is about 1.5 months or less. In some aspects, the response is produced without significantly increasing the risk of capillary leak syndrome (CLS). In some aspects, the response is produced without CLS.

In some aspects, administration of an anti-CD123 immunoconjugate such as pivekimab sunirine produces a CR, CRc, CRh, or CRi, and the time to the CR, CRc, CRh, or CRi is about 0.5 to about 5 months. In some aspects, the time to the complete response is about 0.5 to about 4 months. In some aspects, the time to the CR, CRc, CRh, or CRi is about 0.5 to about 3 months. In some aspects, the time to the CR, CRc, CRh, or CRi is about 0.5 to about 2 months. In some aspects, the time to the CR, CRc, CRh, or CRi is about 0.5 to about 1.5 months.

In some aspects, administration of an anti-CD123 immunoconjugate such as pivekimab sunirine produces a CR or CRc, and the time to the CR or CRc is about 0.5 to about 5 months. In some aspects, the time to the complete response is about 0.5 to about 4 months. In some aspects, the time to the CR or CRc is about 0.5 to about 3 months. In some aspects, the time to the CR or CRc is about 0.5 to about 2 months. In some aspects, the time to the CR or CRc is about 0.5 to about 1.5 months.

In some aspects, administration of an anti-CD123 immunoconjugate such as pivekimab sunirine produces a CR, CRc, CRh, or CRi, and the time to the CR, CRc, CRh, or CRi is about 1 to about 5 months. In some aspects, the time to the complete response is about 1 to about 4 months. In some aspects, the time to the CR, CRc, CRh, or CRi is about 1 to about 3 months. In some aspects, the time to the CR, CRc, CRh, or CRi is about 1 to about 2 months. In some aspects, the time to the CR, CRc, CRh, or CRi is about 1 to about 1.5 months.

In some aspects, administration of an anti-CD123 immunoconjugate such as pivekimab sunirine produces a CR or CRc, and the time to the CR or CRc is about 1 to about 5 months. In some aspects, the time to the complete response is about 1 to about 4 months. In some aspects, the time to the CR or CRc is about 1 to about 3 months. In some aspects, the time to the CR or CRc is about 1 to about 2 months. In some aspects, the time to the CR or CRc is about 1 to about 1.5 months.

In some aspects, the duration of the response, i.e., the CR, CRc, CRh, or CRi is at least 6 months, at least 7 months, at least 8 months, at least 9 months, or at least 10 months. In some aspects, the duration of the response, i.e., the CR, CRc, CRh, or CRi is at least 9 months, at least 10 months, at least 11 months, or at least 12 months. In some aspects, the duration of the response, i.e., the CR, CRc, CRh, or CRi is about 6 months to about 1 year. In some aspects, the duration of the response, i.e., the CR, CRc, CRh, or CRi is about 9 months to about 2 years. In some aspects, the response is produced without significantly increasing the risk of capillary leak syndrome (CLS). In some aspects, the response is produced without CLS.

In some aspects, the duration of the response, i.e., the CR or CRc is at least 6 months, at least 7 months, at least 8 months, at least 9 months, or at least 10 months. In some aspects, the duration of the response, i.e., the CR or CRc is at least 9 months, at least 10 months, at least 11 months, or at least 12 months. In some aspects, the duration of the response, i.e., the CR or CRc is about 6 months to about 1 year. In some aspects, the duration of the response, i.e., the CR or CRc is about 9 months to about 2 years. In some aspects, the response is produced without significantly increasing the risk of capillary leak syndrome (CLS). In some aspects, the response is produced without CLS.

In some aspects, the time to the CR, CRc, CRh, or CRi, in a patient with frontline BPDCN is about 0.5 to about 5 months. In some aspects, the time to the CR, CRc, CRh, or CRi, in a patient with R/R BPDCN is about 0.5 to about 2 months. In some aspects, the time to the CR, CRc, CRh, or CRi, in a patient with R/R BPDCN is about 0.5 to about 5 months. In some aspects, the subject has frontline BPDCN with no prior or concomitant hematological malignancy (PCHM).

In some aspects, the time to the CR or CRc, in a patient with frontline BPDCN is about 0.5 to about 5 months. In some aspects, the time to the CR or CRc, in a patient with R/R BPDCN is about 0.5 to about 2 months. In some aspects, the time to the CR or CRc, in a patient with R/R BPDCN is about 0.5 to about 5 months. In some aspects, the subject has frontline BPDCN with no prior or concomitant hematological malignancy (PCHM).

In some aspects, administration of an anti-CD123 immunoconjugate such as pivekimab sunirine produces a CR, CRc, CRh, CRi, or PR after 3 or less doses of the ant-CD123 immunoconjugate. In some aspects, administration of an anti-CD123 immunoconjugate such as pivekimab sunirine produces a CR, CRc, CRh, CRi, or PR after 1 to 3 doses of the ant-CD123 immunoconjugate. In some aspects, administration of an anti-CD123 immunoconjugate such as pivekimab sunirine produces a CR, CRc, CRh, CRi, or PR after 1 or 2 doses of the anti-CD123 immunoconjugate. In some aspects, the response is produced without significantly increasing the risk of capillary leak syndrome (CLS). In some aspects, the response is produced without CLS.

In some aspects, administration of an anti-CD123 immunoconjugate such as pivekimab sunirine produces a CR or CRc after 3 or less doses of the ant-CD123 immunoconjugate. In some aspects, administration of an anti-CD123 immunoconjugate such as pivekimab sunirine produces a CR or CRc after 1 to 3 doses of the ant-CD123 immunoconjugate. In some aspects, administration of an anti-CD123 immunoconjugate such as pivekimab sunirine produces a CR or CRc after 1 or 2 doses of the anti-CD123 immunoconjugate. In some aspects, the response is produced without significantly increasing the risk of capillary leak syndrome (CLS). In some aspects, the response is produced without CLS.

In some aspects, a patient with BPDCN is eligible for HSCT after achieving a CR, CRc, CRh, or CRi in response to treatment with an anti-CD123 immunoconjugate such as pivekimab sunirine.

In some aspects, a patient with BPDCN is eligible for HSCT after achieving a CR or CRc in response to treatment with an anti-CD123 immunoconjugate such as pivekimab sunirine.

In some aspects, administration of an anti-CD123 immunoconjugate such as pivekimab sunirine produces a CR, CRc, CRh, or CRi after 1 to 3 doses of the ant-CD123 immunoconjugate. In some aspects, administration of an anti-CD123 immunoconjugate such as pivekimab sunirine produces a CR, CRc, CRh, or CRi after 1 or 2 doses of the anti-CD123 immunoconjugate. In some aspects, the response is produced without significantly increasing the risk of capillary leak syndrome (CLS). In some aspects, the response is produced without CLS.

In some aspects, administration of an anti-CD123 immunoconjugate such as pivekimab sunirine produces a CR or CRc after 1 to 3 doses of the ant-CD123 immunoconjugate. In some aspects, administration of an anti-CD123 immunoconjugate such as pivekimab sunirine produces a CR or CRc after 1 or 2 doses of the anti-CD123 immunoconjugate. In some aspects, the response is produced without significantly increasing the risk of capillary leak syndrome (CLS). In some aspects, the response is produced without CLS.

As also demonstrated herein, administration of an anti-CD123 immunoconjugate such as pivekimab sunirine can prepare a subject for a hematopoietic stem cell transplant (HSCT). In some embodiments, administration of an anti-CD123 immunoconjugate such as pivekimab sunirine can prepare at least 30%, at least 35%, at least 40%, at least 45%, or at least 50% of the subjects for a hematopoietic stem cell transplant (HSCT). In some aspects, administration of an anti-CD123 immunoconjugate such as pivekimab sunirine can prepare the subjects for a hematopoietic stem cell transplant without significantly increasing the risk of capillary leak syndrome (CLS). In some aspects, administration of an anti-CD123 immunoconjugate such as pivekimab sunirine can prepare the subjects for a hematopoietic stem cell transplant without CLS.

In some aspects, a patient with BPDCN is eligible for HSCT after 1 to 3 doses of an anti-CD123 immunoconjugate such as pivekimab sunirine. In some aspects, a patient with BPDCN is eligible for HSCT after 1 or 2 doses of an anti-CD123 immunoconjugate such as pivekimab sunirine.

In some aspects, administration of an anti-CD123 immunoconjugate such as pivekimab sunirine improves symptoms in the skin, bone marrow, lymph node, viscera, or a combination thereof in a patient with BPDCN. In some aspects, administration of an anti-CD123 immunoconjugate such as pivekimab sunirine improves symptoms in the skin, bone marrow, lymph node, or a combination thereof in a patient with BPDCN. In some aspects, administration of an anti-CD123 immunoconjugate such as pivekimab sunirine improves the patient's BPDCN-associated symptoms without significantly increasing the risk of capillary leak syndrome (CLS). In some aspects, administration of an anti-CD123 immunoconjugate such as pivekimab sunirine improves the patient's BPDCN-associated symptoms without CLS.

In some aspects, patients receiving an anti-CD123 immunoconjugate such as pivekimab sunirine require red blood cell and/or platelet transfusions prior to the administration of the immunoconjugate. However, as a result of the administration of the immunoconjugate, the subject can become independent of the red blood cell and/or platelet transfusions for at least 28 consecutive days or for at least 56 consecutive days.

### III (D). Premedications

In some aspects, patients receiving an anti-CD123 immunoconjugate as disclosed herein, e.g., pivekimab sunirine, have received pretreatment with a premedication. In some embodiments, the premedication may be a corticosteroid. Accordingly, in some aspects, the methods provided herein comprise administering a corticosteroid to a patient prior to administering an anti-CD123 immunoconjugate, e.g., pivekimab sunirine, to the patient. In some embodiments, the corticosteroid may be administered once, prior to and on the same day as the pivekimab sunirine. In some aspects, the corticosteroid is administered to a patient on the day prior to administering an anti-CD123 immunoconjugate, e.g., pivekimab sunirine, to the patient. In some aspects, the corticosteroid is administered to a patient on the day prior and once, prior to and on the same day as administering an anti-CD123 immunoconjugate, e.g., pivekimab sunirine, to the patient. Accordingly, in some aspects, the corticosteroid is administered to a patient twice on the day prior to administering an anti-CD123 immunoconjugate, e.g., pivekimab sunirine, to the patient. For example, in some aspects, a patient receives two doses of a corticosteroid on the day prior to administering an anti-CD123 immunoconjugate, e.g., pivekimab sunirine. In some embodiments, the corticosteroid may be administered twice on the day prior to administration of the pivekimab sunirine and once on the same day as the pivekimab sunirine, where all administrations of the corticosteroid are administered prior to the pivekimab sunirine. In embodiments, the premedication dosing may provide for less infusion related reactions.

In certain instances, the corticosteroid can be selected from the group consisting of prednisone, prednisolone, methylprednisolone, beclamethasone, betamethasone, dexamethasone, fludrocortisone, hydrocortisone, and triamcinolone. In certain instances the corticosteroid is administered intravenously. In certain instances, the steroid is administered orally.

For example, in some aspects, patients receiving an anti-CD123 immunoconjugate as disclosed herein, e.g., pivekimab sunirine, have received pretreatment with diphenhydramine. In some aspects, patients receiving an anti-CD123 immunoconjugate as disclosed herein, e.g., pivekimab sunirine, have received pretreatment with 25-50 mg diphenhydramine. In some aspects, diphenhydramine is given intravenously. In some aspects, diphenhydramine is given orally. Accordingly, in some aspects, the methods provided herein comprise administering diphenhydramine to a patient prior to administering an anti-CD123 immunoconjugate, e.g., pivekimab sunirine, to the patient.

In some aspects, patients receiving an anti-CD123 immunoconjugate as disclosed herein, e.g., pivekimab sunirine, have received pretreatment with acetaminophen. In some aspects, patients receiving an anti-CD123 immunoconjugate as disclosed herein have received pretreatment with 325-650 mg acetaminophen. In some aspects, acetaminophen is given intravenously. In some aspects, acetaminophen is given orally. Accordingly, in some aspects, the methods provided herein comprise administering acetaminophen to a patient prior to administering an anti-CD123 immunoconjugate, e.g., pivekimab sunirine, to the patient.

In some aspects, patients receiving an anti-CD123 immunoconjugate as disclosed herein, e.g., pivekimab sunirine, have received pretreatment with paracetamol. In some aspects, patients receiving an anti-CD123 immunoconjugate as disclosed herein, e.g., pivekimab sunirine, have received pretreatment with 325-650 mg paracetamol. In some aspects, paracetamol is given intravenously. In some aspects, paracetamol is given orally. Accordingly, in some aspects, the methods provided herein comprise administering paracetamol to a patient prior to administering an anti-CD123 immunoconjugate, e.g., pivekimab sunirine, to the patient.

In some aspects, patients receiving an anti-CD123 immunoconjugate as disclosed herein, e.g., pivekimab sunirine, have received pretreatment with dexamethasone. In some aspects, patients receiving anti-CD123 immunoconjugate as disclosed herein, e.g., pivekimab sunirine, have received pretreatment with 8 mg dexamethasone. In some aspects, patients receiving anti-CD123 immunoconjugate as disclosed herein, e.g., pivekimab sunirine, have received pretreatment with 10 mg dexamethasone. In some aspects, dexamethasone is given intravenously. In some aspects, dexamethasone is given orally. Accordingly, in some aspects, the methods provided herein comprise administering dexamethasone to a patient prior to administering an anti-CD123 immunoconjugate, e.g., pivekimab sunirine, to the patient. Accordingly, in some aspects, the dexamethasone is administered to a patient the day prior to administering an anti-CD123 immunoconjugate, e.g., pivekimab sunirine, to the patient. Accordingly, in some aspects, the dexamethasone is administered to a patient twice on the day prior to administering an anti-CD123 immunoconjugate, e.g., pivekimab sunirine, to the patient. For example, in some aspects, a patient receives two doses of dexamethasone (e.g., two 8 mg doses; two 10 mg doses) on the day prior to administering an anti-CD123 immunoconjugate, e.g., pivekimab sunirine.

In some aspects, patients receiving an anti-CD123 immunoconjugate as disclosed herein, e.g., pivekimab sunirine, have received pretreatment with a diuretic. Accordingly, in some aspects, the methods provided herein comprise administering a diuretic to a patient prior to administering an anti-CD123 immunoconjugate, e.g., pivekimab sunirine, to the patient.

In some aspects, patients receiving an anti-CD123 immunoconjugate disclosed herein, e.g., pivekimab sunirine, also receive a CNS prophylactic treatment. In some aspects, the CNS prophylactic treatment comprises intrathecal chemotherapy.

In some aspects, patients receiving an anti-CD123 immunoconjugate disclosed herein, e.g., pivekimab sunirine, also receive ursodeoxycholic acid.

In some aspects, patients receiving an anti-CD123 immunoconjugate disclosed herein, e.g., pivekimab sunirine, also receive diuretic treatment. Accordingly, in some aspects, the methods provided herein comprise administering a diuretic to a patient after administering an anti-CD123 immunoconjugate, e.g., pivekimab sunirine, to the patient.

Aspects of the present disclosure can be further defined by reference to the following non-limiting examples. It will be apparent to those skilled in the art that many modifications, both to materials and methods, can be practiced without departing from the scope of the present disclosure.

### Examples

It is understood that the examples and aspects described herein are for illustrative purposes only and that various modifications or changes in light thereof will be suggested to persons skilled in the art and are to be included within the spirit and purview of this application

### Example 1: Treatment of BPDCN in Human Patients Using Pivekimab Sunirine

Patients with BPDCN were enrolled into a phase 2 clinical trial of pivekimab sunirine in one of the following cohorts:
Cohort 1: Patients with relapsed or refractory (R/R) BPDCN who have received 1-3 prior systemic therapies (including tagraxofusp-erzs and any other systemic therapy deemed appropriate for the treatment of BPDCN)
Cohort 2: Patients with frontline de novo BPDCN who have not received prior systemic therapy and patients with frontline BPDCN who have a prior or concomitant hematologic malignancy (PCHM) and have not received prior systemic therapy

Key inclusion and exclusion criteria were as follows.

### Key Inclusion Criteria

Patients ≥18 years old
Frontline or R/R BPDCN
Confirmation of CD123 positivity on flow cytometry or immunohistochemistry (IHC). Patients who received prior CD123-targeting agents were allowed as long as the blasts still had detectable CD123 expression.
Frontline patients in Cohort 2 may have received local therapy (e.g., radiotherapy, surgical excision, photodynamic therapy)
Eastern Cooperative Oncology Group (ECOG) performance status ≥1. If nonambulatory due to a chronic disability, Karnofsky performance status was >70.
Previous treatment-related toxicities must have been resolved to Grade 1 (excluding alopecia).
Liver enzymes (aspartate aminotransferase and alanine aminotransferase)≤2.5× the upper limit of normal (ULN). Exceptions could be made for patients with elevated liver transaminases secondary to the underlying study disease.
Total bilirubin ≤1.5×ULN; patients with Gilbert syndrome had have total bilirubin <3.0×ULN with direct bilirubin <1.0×ULN at the time of enrollment.
Estimated glomerular filtration rate of >30 mL/min/1.73 m² or creatinine clearance of >30 mL/min.
Left ventricular ejection fraction ≥45%.
Patients with a prior autologous or allogeneic bone marrow transplant were eligible for Cohorts 1 and 2. Patients with an allogeneic transplant met the following conditions: the transplant must have been performed more than 120 days before the date of dosing on this study, the patient must not have active ≥Grade 2 acute graft versus host disease (GvHD), or extensive chronic GvHD of any severity, and must be off all immunosuppression for at least 2 weeks before first dose of pivekimab sunirine.
Patients with prior malignancy were eligible. Patients with a PCHM were eligible as long as no current therapy was still required for the second malignancy (e.g., MDS, CMML). Patients with a nonhematologic prior malignancy were in remission from the prior malignancy. Patients had completed all chemotherapy and radiotherapy for prior malignancy at least 6 months before enrollment and all treatment-related toxicities must have resolved to Grade 1 or less.
Patients with prostate cancer or breast cancer on adjuvant hormonal therapy were eligible.
Patients with tumors with a negligible risk for metastasis or death (e.g., adequately controlled basal cell carcinoma or squamous cell carcinoma of the skin, or carcinoma in situ of the cervix or breast) were eligible.

### Key Exclusion Criteria

Frontline BPDCN patients with central nervous system (CNS) disease were excluded. A lumbar puncture was required during the 28-day Screening period, before drug administration. Relapsed or refractory BPDCN patients with a known history of CNS disease must have been treated locally, have at least 1 lumbar puncture with no evidence of CNS disease, and must have been clinically stable before first dose. Concurrent therapy for CNS prophylaxis or continuation of therapy for controlled CNS disease was encouraged.
Patients with a history of veno-occlusive disease of the liver.
Patients with a history of Grade 4 capillary leak syndrome, or noncardiac Grade 4 edema are ineligible, e.g., related to tagraxofusp-erzs or other etiology.
Corrected QT interval (QT interval corrected using Fridericia's formula)>480 msec.
Myocardial infarction within 6 months before enrollment or has New York Heart Association Class III or IV heart failure, uncontrolled angina, severe uncontrolled ventricular arrhythmias, or electrocardiogramavidence of acute ischemia or active conduction system abnormalities before study entry.
Interval from prior cancer therapy:
   a) For frontline BPDCN patients with prior local therapy (e.g., radiotherapy), patients must not have received treatment within 14 days before drug administration on this study.
   b) Relapsed or refractory BPDCN patients must not have received any anticancer therapy including chemotherapy, immunotherapy, radiotherapy, hormonal, biologic, or any investigational agents within 14 days before drug administration on this study. Patients must have recovered to baseline from all acute toxicity from this prior therapy.
   c) Patients who have received a checkpoint inhibitor must not have received that therapy within 28 days before drug administration on this study.
Clinically relevant active infection including known active hepatitis B or C, human immunodeficiency virus infection, or cytomegalovirus or any other known concurrent infectious disease that, in the judgment of the Investigator, would make a patient inappropriate for enrollment into this study (testing not required).
Patients who have undergone a major surgery within 4 weeks (or longer if not fully recovered) before study enrollment.
Serious or poorly controlled medical conditions that could be exacerbated by treatment or that would seriously compromise safety assessment or compliance with the protocol, in the judgment of the Investigator.
Patients who have a history of allergy to pivekimab sunirine or any of its excipients.
Patients who received a live vaccine four weeks or fewer prior to enrollment.

A total of 30 patients with frontline BPDCN and 49 patients with R/R BPDCN were enrolled. The characteristics of these patients are listed in Table 4.

TABLE 4
BPDCN Patient Characteristics for Interim analysis

| | | Frontline | R/R |
|---|---|---|---|
| | | BPDCN | BPDCN |
| | | N = 30 | N = 49 |
| Age, % | Median (range), y | 74 (48-84) | 69 (19-82) |
| | ≥65 y | 90 | 59 |
| Gender, % | Male | 80 | 82 |
| | Female | 20 | 18 |
| Disease, % | Skin | 93 | 65 |
| | Bone marrow | 43 | 47 |
| | Nodal disease (PET/CT scan) | 47 | 41 |
| Baseline | Primary refractory | — | 43 |
| status, % | First relapse | — | 33 |
| | Second or greater relapse | — | 25 |
| Prior | Non-intense (e.g., | — | 74 |
| therapy, % | VEN +/− HMA, CVP, | | |
| | steroids) | | |
| | Tagraxofusp | — | 43 |
| | Intense | — | 55 |
| | (e.g. HyperCVAD, FLAG, | | |
| | CHOP) | | |
| | Stem cell transplant | — | 33 |
| | (14 allo, 2 auto) | | |
| | Local radiotherapy | 7 | 18 |
| Number of | 1 | 1 | 53 |
| prior lines of | 2 | — | 27 |
| treatment | 3+ | — | 18 |

TABLE 5
Final Frontline BPDCN Patient Characteristics. Primary Analysis
Population (PAP): All frontline BPDCN patients without prior
systemic therapy and no prior or concomitant hematologic malignancy
(PCHM) on screening bone marrow assessment.

| | | Total |
|---|---|---|
| | | Frontline |
| | PAP | BPDCN |
| | (1 L de | (de novo + |
| | novo BPDCN) | PCHM) |
| Number of Patients, n (%) | (N = 20) | (N = 33)* |

Age (years)

| n | 20 | | 33 | |
|---|---|---|---|---|
| Median (range) | 73.5 | (48, 84) | 73.0 | (48, 84) |

Age categories (years) - n (%)

| | | | | |
|---|---|---|---|---|
| 18-<65 | 3 | (15.0) | 3 | (9.1) |
| 65-<75 | 10 | (50.0) | 17 | (51.5) |
| >=65 | 17 | (85.0) | 30 | (90.9) |
| >=75 | 7 | (35.0) | 13 | (39.4) |

Sex - n (%)

| | | | | |
|---|---|---|---|---|
| Male | 17 | (85.0) | 27 | (81.8) |
| Female | 3 | (15.0) | 6 | (18.2) |

Race - n (%)

| | | | | |
|---|---|---|---|---|
| White | 15 | (75.0) | 27 | (81.8) |
| Black | 1 | (5.0) | 1 | (3.0) |
| Asian | 0 | (0) | 0 | (0) |
| Not reported | 4 | (20.0) | 5 | (15.2) |

Ethnicity - n (%)

| | | | | |
|---|---|---|---|---|
| Hispanic or Latino | 3 | (15.0) | 4 | (12.1) |
| Not Hispanic or Latina | 17 | (85.0) | 28 | (84.8) |
| Missing | 0 | (0) | 1 | (3.0) |

ECOG Performance Status - n (%)

| | | | | |
|---|---|---|---|---|
| 0 | 7 | (35.0) | 12 | (36.4) |
| 1 | 13 | (65.0) | 21 | (63.6) |

| | | |
|---|---|---|
| 2 | 0 | 0 |
| 3 | 0 | 0 |

Bone marrow blast count - n (%)

| | | | | |
|---|---|---|---|---|
| <=5% | 11 | (55.0) | 17 | (51.5) |
| >5% | 9 | (45.0) | 16 | (48.5) |

| | | |
|---|---|---|
| Missing | 0 | 0 |

Bone marrow blast count (%)

| n | 20 | | 33 | |
|---|---|---|---|---|
| Mean (SD) | 20.1 | (27.64) | 21.8 | (27.87) |
| Median (Range) | 5.0 | (0, 89) | 5.0 | (0, 89) |

Baseline Disease Involvement - n (%)

| | | | | |
|---|---|---|---|---|
| Skin | 18 | (90.0) | 31 | (93.9) |
| Bone Marrow | 9 | (45.0) | 16 | (48.5) |
| Peripheral Blood | 1 | (5.0) | 3 | (9.1) |
| Lymph Nodes | 6 | (30.0) | 12 | (36.4) |

| | | |
|---|---|---|
| Viscera | 0 | 0 |

Type of BPDCN - n (%)

| | | | | |
|---|---|---|---|---|
| De novo | 20 | (100.0) | 22 | (66.7) |

| | | | |
|---|---|---|---|
| Secondary | 0 | 11 | (33.3) |

Type of secondary BPDCN - n (%)

| Antecedent Malignancy | 0 | | 11 | (33.3) |
|---|---|---|---|---|
| Treatment-related | 0 | | 0 | |
| Missing | 20 | (100.0) | 22 | (66.7) |

Baseline Hepatic Function - n (%)

| | | | | |
|---|---|---|---|---|
| Normal | 16 | (80.0) | 27 | (81.8) |
| Mild Impairment | 3 | (15.0) | 4 | (12.1) |
| Moderate Impairment | 1 | (5.0) | 2 | (6.1) |

Baseline Renal Function - n (%)

| | | | | |
|---|---|---|---|---|
| Normal | 8 | (40.0) | 13 | (39.4) |
| Mild Impairment | 12 | (60.0) | 18 | (54.5) |

| | | | |
|---|---|---|---|
| Moderate Impairment | 0 | 2 | (6.1) |

TABLE 6
Final R/R BPDCN Patient Characteristics

| | |
|---|---|
| | R/R |
| | BPDCN |
| Number of Patients, n (%) | (N = 51) |

Age (years)

| n | 51 | |
|---|---|---|
| Median (range) | 69.0 | (19, 85) |

Age categories (years) - n (%)

| | | |
|---|---|---|
| 18-<65 | 21 | (41.2) |
| 65-<75 | 16 | (31.4) |
| >=65 | 30 | (58.8) |
| >=75 | 14 | (27.5) |

Sex - n (%)

| | | |
|---|---|---|
| Male | 42 | (82.4) |
| Female | 9 | (17.6) |

Race

| | | |
|---|---|---|
| White | 42 | (82.4) |
| Black | 2 | (3.9) |
| Asian | 1 | (2.0) |
| Not reported | 6 | (11.8) |

Ethnicity

| | | |
|---|---|---|
| Hispanic or Latino | 8 | (15.7) |
| Not Hispanic or Latino | 38 | (74.5) |
| Missing | 5 | (9.80) |

ECOG Performance Status - n (%)

| | | |
|---|---|---|
| 0 | 18 | (35.3) |
| 1 | 30 | (58.8) |
| 2 | 2 | (3.9) |
| 3 | 1 | (2.0) |

Bone marrow blast count - n (%)

| | | |
|---|---|---|
| <=5% | 24 | (47.1) |
| >5% | 24 | (47.1) |
| Missing | 3 | (5.9) |

Bone marrow blast count (%)

| n | 48 | |
|---|---|---|
| Mean (SD) | 26.0 | (32.68) |
| Median (Range) | 6.0 | (0.99) |

Baseline Disease Involvement - n (%)

| | | |
|---|---|---|
| Skin | 34 | (66.7) |
| Bone Marrow | 24 | (47.1) |
| Peripheral Blood | 10 | (19.6) |
| Lymph Nodes | 18 | (35.3) |
| Viscera | 3 | (5.9) |

Type of BPDCN - n (%)

| | | |
|---|---|---|
| De novo | 42 | (82.4) |
| Secondary | 9 | (17.6) |

Type of secondary BPDCN - n (%)

| | | |
|---|---|---|
| Antecedent Malignancy | 7 | (13.7) |
| Treatment-related | 2 | (3.9) |
| Missing | 42 | (82.4) |

Baseline Hepatic Function - n (%)

| | | |
|---|---|---|
| Normal | 43 | (84.3) |
| Mild Impairment | 6 | (11.8) |
| Moderate Impairment | 2 | (3.9) |

Baseline Renal Function - n (%)

| | | |
|---|---|---|
| Normal | 26 | (51.0) |
| Mild Impairment | 20 | (39.2) |
| Moderate Impairment | 5 | (9.8) |

Patients received a 0.045 mg/kg intravenous dose of pivekimab sunirine on day 1 of a 21-day cycle as a<30-minute outpatient infusion. Lyophilized pivekimab sunirine was reconstituted using 5.4 mL of water for injection. Planned treatment consisted of 2 cycles (6 weeks). Additional cycles, up to 10 total, were administered for patients deriving benefit from this regimen. CNS prophylactic intrathecal chemotherapy was administered. In particular, for relapsed or refractory patients with CNS positivity at screening (and subsequently at least one negative LP), aggressive CNS-directed intrathecal chemotherapy was used: intrathecal treatments at least weekly for at least 4 doses; then, twice per month for a total of at least 8 doses; and then, optionally, once or twice per month. For patients without CNS positivity, prophylactic CNS-directed intrathecal chemotherapy was strongly recommended: intrathecal treatments twice per month for a total of at least 8 doses; and then, optionally, once or twice per month. Chemotherapy regimens followed institutional standards, but generally were aggressive including alternating cytarabine with methotrexate, or triple intrathecals (cytarabine, methotrexate, steroid).

Patients received 8 mg dexamethasone on the day before any pivekimab sunirine dose. Patients also received 25-50 mg diphenhydramine, 325-650 mg acetaminophen or paracetamol, and 8 mg dexamethasone before each pivekimab sunirine infusion.

Following preparation, IMGN632 (or pivekimab sunirine (pvek)) was administered by IV syringe pump. Overall length of infusion was varied depending on dose and patient tolerability. Initially, the study drug was administered at a rate of 0.165 mg/min (0.8 mL/min); and, after 30 minutes, if well tolerated, the rate was increased to 0.33 mg/min. Subsequent infusions were delivered at the highest tolerated rate. Before and following infusion, the line was flushed with 5% dextrose to ensure delivery of the full dose. Patients were carefully observed during each infusion and vital signs were monitored. Patients remained in the clinic under observation for 4 hours after the first infusion and for at least 1 hour after each subsequent infusion.

At the interim analysis, tolerable safety results were observed in both frontline and R/R BPDCN patients. The safety profile was manageable with mostly low-grade peripheral edema and infusion-related reactions. The discontinuations due to pivekimab sunirine-related adverse events were 3%. The 30-day mortality rate was 0% in frontline patients and 4% in R/R patients (2 deaths due to disease progression). Interim safety results from all frontline and R/R BPDCN patients (n=79) are summarized in Table 5.

TABLE 7
Interim Safety Overview

| Treatment Emergent | All | |
|---|---|---|
| Adverse Events (TEAE) | Grades ≥15% | ≥3 Grade |
| Peripheral edema | 46% | 10% |
| Thrombocytopenia | 27% | 19% |
| Fatigue | 35% | 4% |
| Infusion-related reactions (IRRs) | 25% | 4% |
| Constipation | 23% | 0% |
| Nausea | 22% | 0% |
| Anemia | 20% | 8% |
| Headache | 19% | 4% |
| Neutropenia | 18% | 17% |
| Diarrhea | 17% | 0% |
| Hypokalemia | 17% | 3% |
| Dyspnea | 15% | 1% |
| Hyperglycemia | 15% | 6% |
| Pyrexia | 15% | 1% |

At the interim analysis, less frequent than the adverse events included liver-related adverse events. ALT (3%), AST (3%) and TBILI (1%) laboratory elevations of grade 3 were observed, but there were no grade 4 or 5 events. One dose limiting toxicity (DLT), a Grade 3 ALT with a duration of 7 days, was observed. Reversible VOD of all grades were observed in 4% (3/79) of patients, with grade 3 in 1% (n=1). There were no grade 4 or 5 VOD events. One patients proceeded to transplant without hepatic complications. Hypoalbuminemia of all grades was observed in 13% of patients, with grade 3 in 3% (n=2). There were no grade 4 or 5 hypoalbuminemia events. No capillary leak syndrome (CLS) events were reported.

At the final analysis, pivekimab sunirine demonstrated a favorable safety profile across all BPDCN patients with few treatment-related discontinuations. Risks with pivekimab sunirine include edema, Infusion-related reactions (IRRs), and veno-occlusive disease (VOD). IRRs and edema were predominantly of low grade and manageable. VOD risk is higher in post-hematopoietic stem cell transplant (HSCT), particularly in R/R BPDCN. No capillary leak syndrome (CLS) or treatment-related deaths were reported. The safety risks were controlled in the clinical trial by close monitoring and guidelines for dose modification and supportive care.

TABLE 8

| | |
|---|---|
| Total Frontline | |
| BPDCN | R/R |
| (de novo + PCHM) | BPDCN |
| (N = 33) | (N = 51) |

Final Safety Overview - TEAEs (≥15% of patients)

| | | | | |
|---|---|---|---|---|
| Patients with any TEAE | 33 | (100.0) | 50 | (98.0) |
| Oedema peripheral | 24 | (72.7) | 21 | (41.2) |
| Fatigue | 10 | (30.3) | 12 | (23.5) |
| Infusion related reaction | 3 | (9.1) | 19 | (37.3) |
| Diarrhea | 7 | (21.2) | 8 | (15.7) |
| Nausea | 7 | (21.2) | 10 | (19.6) |
| Constipation | 6 | (18.2) | 10 | (19.6) |
| Anaemia | 9 | (27.3) | 7 | (13.7) |
| Hypokalaemia | 10 | (30.3) | 7 | (13.7) |
| Thrombocytopenia | 4 | (12.1) | 12 | (23.5) |
| Headache | 9 | (27.3) | 5 | (9.8) |
| Pyrexia | 3 | (9.1) | 8 | (15.7) |

| | | | |
|---|---|---|---|
| Veno-occlusive disease | 2 | (6.1) | 0 |

Final Safety Overview - Grade ≥3 TEAEs (≥5% of patients)

| Patients with any Grade ≥3 TEAEs | 28 | (84.8) | 38 | (74.5) |
|---|---|---|---|---|
| Thrombocytopenia | 3 | (9.1) | 9 | (17.6) |
| Febrile neutropenia | 1 | (3.0) | 4 | (7.8) |
| Neutropenia | 5 | (15.2) | 8 | (15.7) |
| Anaemia | 3 | (9.1) | 4 | (7.8) |
| Oedema peripheral | 8 | (24.2) | 2 | (3.9) |
| Pneumonia | 2 | (6.1) | 4 | (7.8) |
| Platelet count decreased | 5 | (15.2) | 3 | (5.9) |
| White blood cell count decreased | 2 | (6.1) | 5 | (9.8) |
| Hyperglycaemia | 1 | (3.0) | 3 | (5.9) |
| Infusion related reaction | 0 | | 3 | (5.9) |
| Leukocytosis | 2 | (6.1) | 1 | (2.0) |

Safety profile for pivekimab sunirine monotherapy is manageable and tolerable and few treatment-related discontinuations. Risks with pivekimab sunirine include VOD, IRR and edema. Analyses of edema events correlating to identify capillary leak syndrome (CLS) with pivekimab sunirine were conducted and were not associated with CLS. The safety risks were controlled in the clinical trial by close monitoring and guidelines for dose modification and supportive care. As pivekimab sunirine is an antibody therapy, IRRs were anticipated. In general, pivekimab sunirine has been well-tolerated with IRRs being mild to moderate, of short duration, and reversible with prophylaxis and supportive care. Edema is the most frequent adverse reaction reported with pivekimab sunirine administration. The most commonly reported event is peripheral edema in 52.6% (61/116) of patients, with 19.8% (23/116) Grade 1, 16.4% (19/116) Grade 2, and 16.4% (19/116) Grade ≥3 in severity. Overall, events of edema were managed with early initiation of diuretics and appropriate dose adjustments with low drug-discontinuation rates. VOD is a known side effect with DNA damaging ADC drugs like MYLOTARG® (gemtuzumab ozogamicin) and BESPONSA® (inotuzumab ozogamicin). Veno-occlusive disease has been reported in subjects receiving pivekimab Sunirine while on treatment as well as in patients after completing the treatment and receiving consolidative HSCT. 2 of 84 (2.4%) patients experienced on treatment VOD and both (1: Grade 2 and 1: Grade 3) are frontline BPDCN patients. Among 5 patients with post-HCT VOD; 1 in 1L BPDCN (Unknown Grade) and 4 in R/R BPDCN (2: Grade 4, 1: Grade 5, 1: unknown Grade).

TABLE 9
Safety summaries from pivekimab sunirine and tagraxofusp
(ELZONRIS ®) monotherapy in BPDCN patients

| | |
|---|---|
| All Grade | Grade ≥3 |

Pivekimab Sunirine All Patients at 0.045
mg/kg once every 3 weeks (N = 116)

| Infusion Related | 26% (n = 30) | 5% (n = 6) |
|---|---|---|
| Reaction | 4.3% (on or after | |
| | additional premed) | |
| Capillary Leak | Not Reported | |
| Syndrome | | |
| Generalized Edema | 4% (n = 4) | 3% (n = 3) |
| Peripheral Edema | 45% (n = 52) | 9% (n = 10) |
| Liver Enzymes | 10.3% (n = 12)/ | 3% (n = 3)/ |
| Elevation (ALT/AST) | 8.6% (n = 10) | 2% (n = 2) |
| On Treatment (TEAE) | 2% (n = 2) | 1% (n = 1) |
| VOD | | |
| Post-HSCT VOD | 26.3% (n/N* = 5/19) | 15.8% (n/N * = 3/19) |
| | (2 with G4, 1 with | (2 with G4, 1 |
| | G5, 2 unknown) | with G5) |

Elzonris All Patients (N = 122)

| Infusion Related | 43%\hat{ } (n = 53) | 10% (n = 7) |
|---|---|---|
| Reaction | | |
| Capillary Leak | 53% (n = 65) | 11% (n = 12) |
| Syndrome | | |
| Generalized Edema | Not Reported | |
| Peripheral Edema | 39% | 1% |
| Liver Enzymes | 79% (n = 16)/ | 26% (n = 32)/ |
| Elevation (ALT/AST) | 76% (n = 93) | 33% (n = 36) |
| On Treatment (TEAE) | Not Reported | |
| VOD | | |

| | |
|---|---|
| Post-HSCT VOD | Not Reported |

\hat{ }Hypersensitive reaction;
*BPDCN patients receiving HSCT

All three areas of potential disease involvement (skin, bone marrow, nodal/visceral) were assessed to establish a response. Subsequent repeated computed tomography/positron emission tomography (CT/PET) scans were performed on patients with nodal/visceral disease at baseline, or as indicated clinically. Bone marrow and skin assessments continued for all patients. Response assessments by bone marrow aspirates (BMAs) for differential assessments were made at the end of Cycles 1, 2, 4, and 6, then every 4 cycles thereafter (approximately every 3 months). Nodal/visceral involvement was assessed by CT/PET after the end of Cycles 2, 4, and 6, and at the end of every 4 cycles thereafter. Skin involvement was assessed by photographs and Modified Severity-Weighted Assessment Tool (mSWAT) at the end of Cycle 1 and 2, and at the end of every 2 cycles thereafter (approximately every 6 weeks). The primary endpoints of the study were rates of complete response (CR) and clinical complete response (CRc). The key secondary endpoints were overall response rate (ORR; CR+CRc+CRh (CR with partial hematologic recovery)+CRi (CR with incomplete recovery)+PR (partial response)) and duration of overall response (DOR).

TABLE 10
Response criteria for BPDCN

| Response | Location | Criteria |
|---|---|---|
| Complete Response | Marrow | Normalization of blast percentage (≤5%) |
| (CR) | Peripheral | Normalization of neutrophil count (≥1000/μL) |
| | Blood | and platelet count (≥100,000/μL) |
| | | Absence of leukemic blasts |
| | Skin | 100% clearance of all skin lesions from baseline; |
| | | no new lesions in patients without lesions at |
| | | baseline |
| | Nodal Masses | Regression to normal size on computed |
| | | tomography (CT) |
| | Spleen, Liver | Not palpable, nodules disappeared |
| CR (clinical) with | Marrow | Normalization of blast percentage (≤5%) |
| minimal residual skin | | |
| abnormality (CRc) | | |
| | Peripheral | Normalization of neutrophil count (≥1000/μL) |
| | Blood | and platelet count (≥100,000/μL) |
| | | Absence of leukemic blasts |
| | Skin | Marked clearance of all skin lesions from |
| | | baseline; residual hyperpigmentation or |
| | | abnormality with BPDCN identified on biopsy |
| | | (or no biopsy performed) |
| | Nodal Masses | Regression to normal size on CT |
| | Spleen, Liver | Not palpable, nodules disappeared |
| CR (clinical) with | Marrow | Normalization of blast percentage (≤5%) |
| partial hematologic | | |
| recovery (CRh) | | |
| | Peripheral | Normalization of neutrophil count (≥500/μL) |
| | Blood | AND platelet count (≥50,000/μL) |
| | | Absence of leukemic blasts |
| | Skin | Marked clearance of all skin lesions from |
| | | baseline; residual hyperpigmentation or |
| | | abnormality with BPDCN identified on biopsy |
| | | (or no biopsy performed) |
| | Nodal Masses | Regression to normal size on CT |
| | Spleen, Liver | Not palpable, nodules disappeared |
| CR (clinical) | Marrow | Normalization of blast percentage (≤5%) |
| with incomplete | | |
| recovery (CRi) | | |
| | Peripheral | Normalization of neutrophil count (≥1000/μL) |
| | Blood | OR platelet count (≥100,000/μL) |
| | | Absence of leukemic blasts |
| | Skin | Marked clearance of all skin lesions from |
| | | baseline; residual hyperpigmentation or |
| | | abnormality with BPDCN identified on biopsy |
| | | (or no biopsy performed) |
| | Nodal Masses | Regression to normal size on CT |
| | Spleen, Liver | Not palpable, nodules disappeared |
| Partial | Marrow | Decreased by >50% in blast percentage to 5%- |
| Response (PR) | | 25% |
| | Skin | 50% to <100% clearance of all skin lesions from |
| | | baseline; no new lesions in patients without |
| | | lesions at baseline |
| | Nodal Masses | ≥50% decrease in the sum of the product of the |
| | | diameters (SPD) of up to 6 largest dominant |
| | | masses; no increase in size of other nodes |
| | Spleen, Liver | ≥50% decrease in SPD of nodules (for single |
| | | nodule in greatest transverse diameter); no |
| | | increase in size of liver of spleen |
| Stable Disease (SD) | | Failure to achieve at least a PR, but no evidence |
| | | of progression for at least 8 weeks |
| Relapse after | Marrow | Blast percentage >5% (if no peripheral blasts, |
| CR/CRi/CRc | | then confirmation aspirate required ≥1 week |
| | | later) |
| | Peripheral | Presence of leukemic blasts |
| | Blood | |
| | Skin | Increase in skin score greater than the sum of |
| | | nadir plus 50% baseline score |
| | Nodal Masses | Appearance of a new lesion(s) >1.5 cm in any |
| | | axis, ≥50% increase from nadir in SPD of more |
| | | than 1 node, or ≥50% increase from nadir in |
| | | longest diameter of a previously identified |
| | | node >1 cm in short axis |
| | Spleen, Liver | > 0% increase from nadir in the SPD of any |
| | | previous lesions |
| Progressive Disease | Marrow | Any new lymph nodes or new skin lesions OR |
| (PD) | | increase from nadir by >50% of SPD of any |
| | Nodal Masses | single previously involved lymph node or total |
| | Skin | assessed lymph node masses OR >50% increase |
| | | in bone marrow blast percentage |

Hematologic parameters, i.e., neutrophil and platelet counts, could be assessed up to 14 days following bone marrow assessment to make formal response criteria assessment.

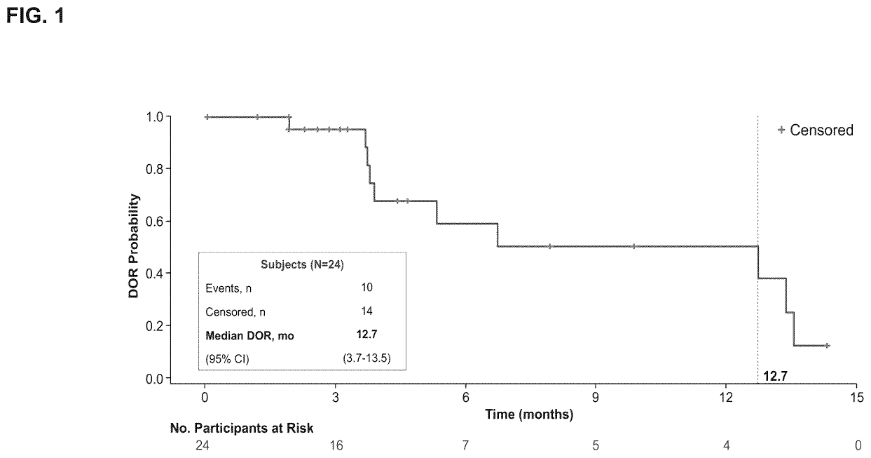

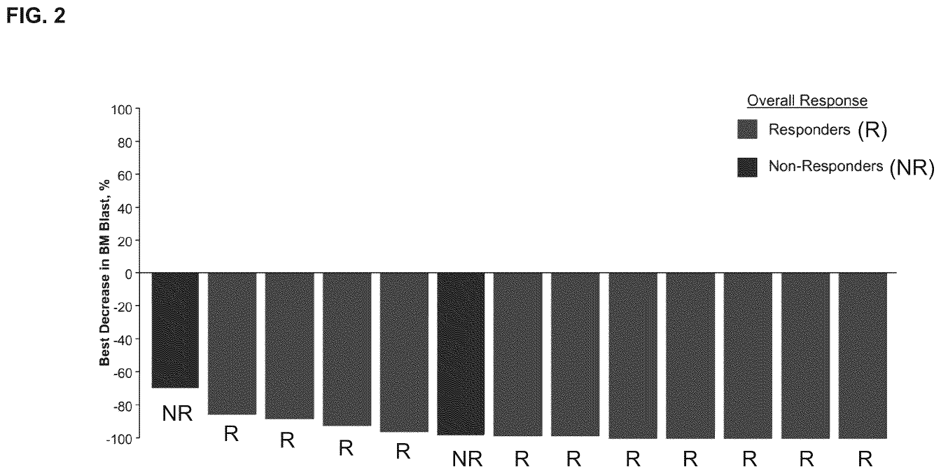

Interim response data for frontline BPDCN patients is shown in Table 11. Of this patient group, 9 patients (30%) were bridged to allogeneic stem cell transplant. The median duration of response (DOR) in frontline BPDCN patients was 12.7 months with a 95% confidence interval of 3.7-13.5 months (FIG. 1). In frontline BPDCN patients with bone marrow involvement at baseline (>5% bone marrow blasts), 100% (13/13) of patients achieved bone marrow remission (<5% blasts). Of these, 11 out of 13 patients achieved an overall response (FIG. 2).

TABLE 11
Interim response data in frontline BPDCN patients

| | |
|---|---|
| ORR | Composite CR |

| | | | | |
|---|---|---|---|---|
| Response rate | 80% | (24/30) | 73% | (22/30) |

Time to first response

| | | | | |
|---|---|---|---|---|
| Median (range), months | 1.3 | (0.5-3.5) | 1.5 | (0.5-4.6) |

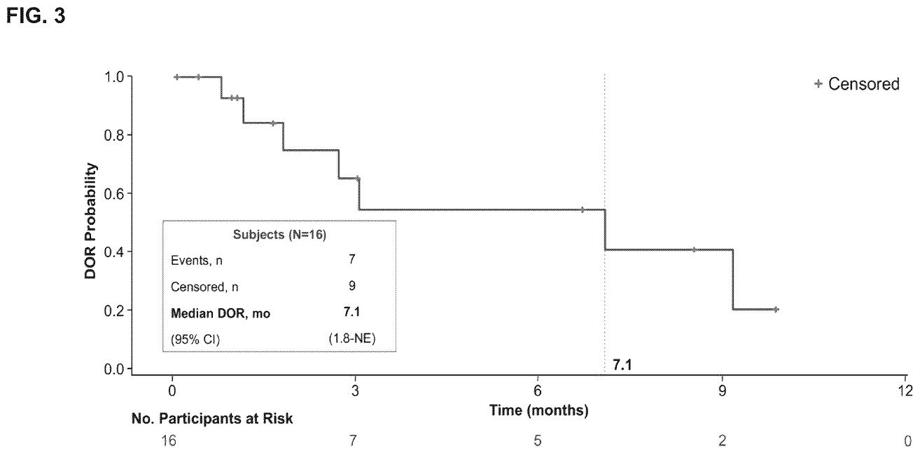

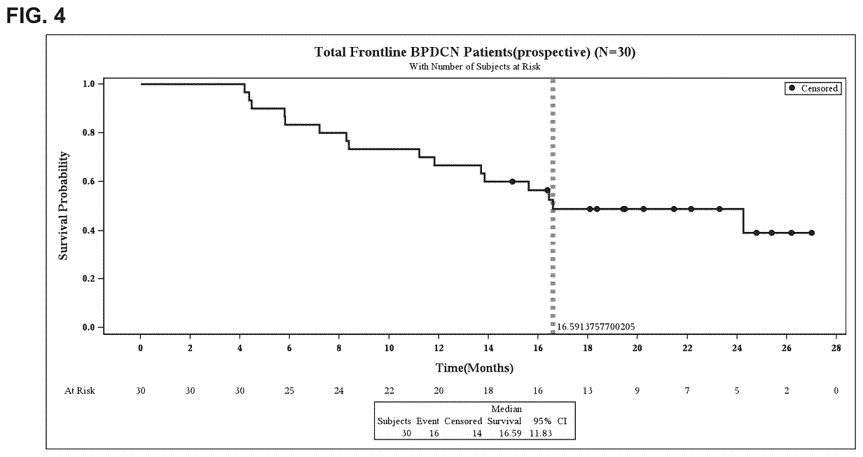

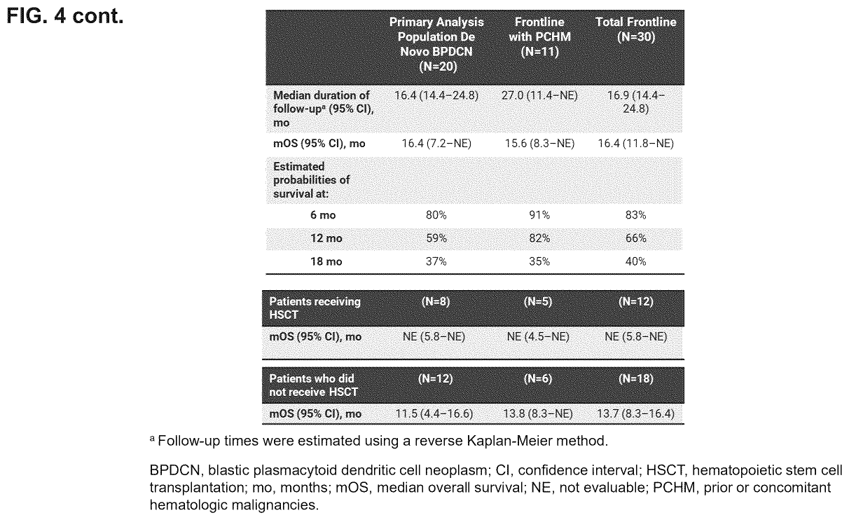

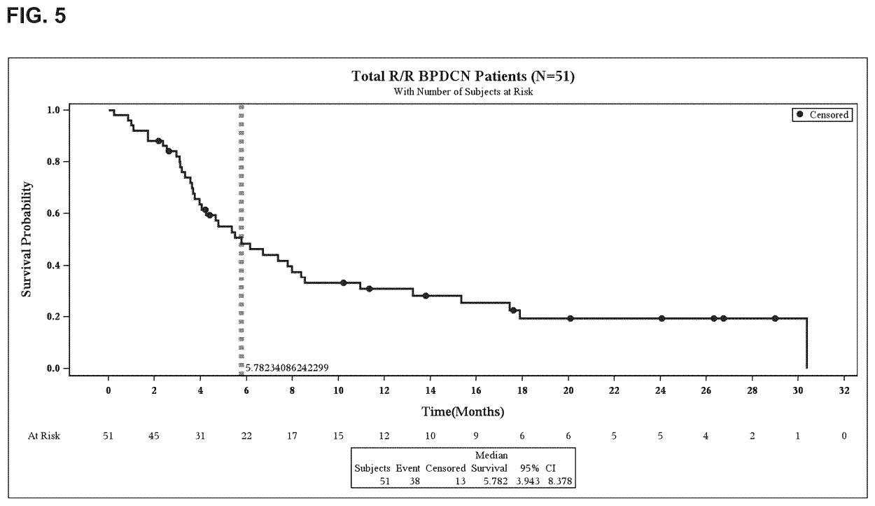

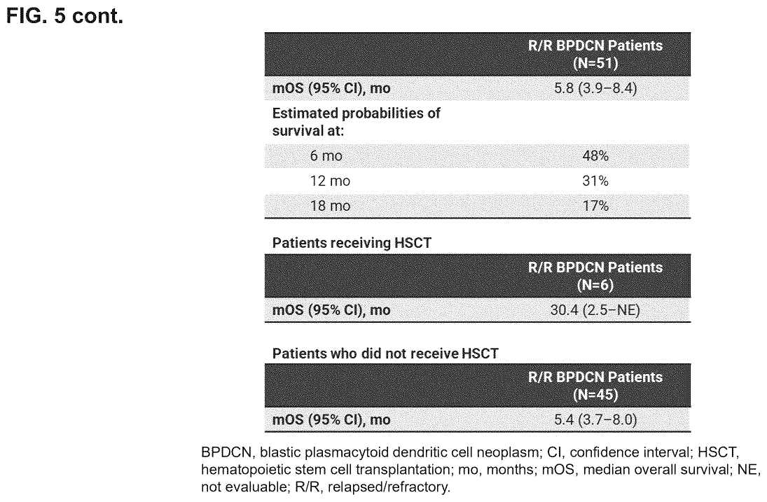

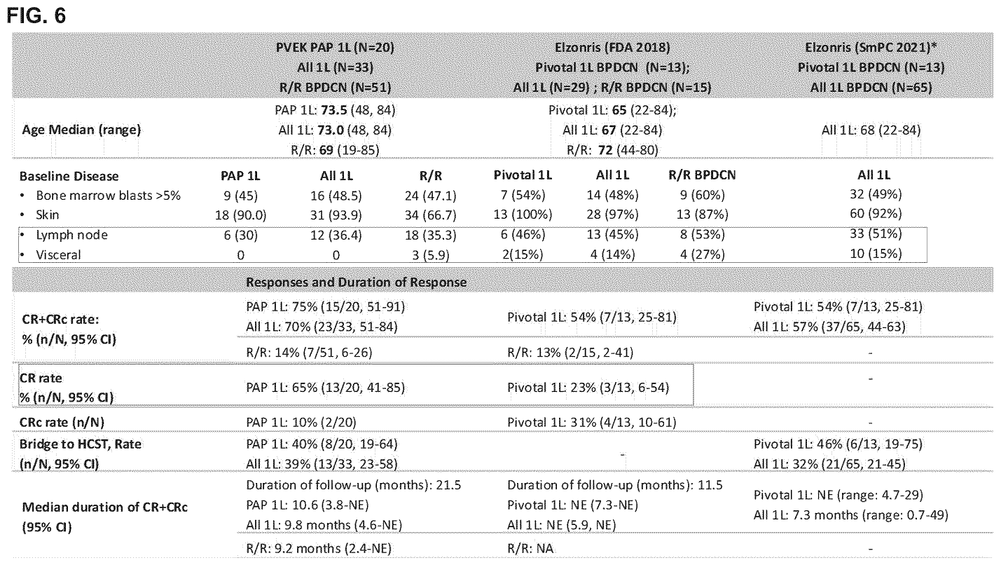

Interim response data for R/R BPDCN patients is shown in Table 12. Of this patient group, 4 patients (8%) were bridged to allogeneic stem cell transplant. In the 10 patients who did not respond to prior tagraxofusp, 3 of the 10 had a response to pivekimab sunirine. The median duration of response in R/R BPDCN patients was 7.1 months (FIG. 3).

TABLE 12
Interim response data in R/R BPDCN patients

| | | | Composite | |
|---|---|---|---|---|
| | ORR | | CR | |
| All R/R patients | 33% | (16/49) | 20% | (10/49) |
| Patients who received prior | 33% | (7/21) | 19% | (4/21) |
| tagraxofusp (n = 21) | | | | |
| Patients who had prior | 50% | (8/16) | 31% | (5/16) |
| SCT (n = 16) | | | | |

Time to first response in all R/R patients

| | | | | |
|---|---|---|---|---|
| Median (range), months | 1.3 | (0.6-3.7) | 1.4 | (0.7-1.9) |

Pivekimab sunirine monotherapy led to high composite CR rates (73%) in frontline BPDCN, as well as durable responses in R/R patients (DOR 7.1 months), including those treated with prior tagraxofusp. Pivekimab sunirine demonstrates compelling and surprising clinical activity and tolerability in both frontline and R/R BPDCN.

In the final analysis, the study results demonstrate a favorable benefit-risk profile of pivekimab sunirine in patients with BPDCN Primary endpoint of composite complete response (CR+CRc (Complete response+clinical response with limited residual skin disease)) rate of 75% (95% CI: 50.9, 91.3) in PAP exceeding the statistical benchmark (95% CI lower bound of 51%, ruling out a 10% null hypothesis) was observed. Median duration of follow-up 21.5 months (range 18.4-24.8) and estimated probability of survival at 18 months for all frontline participants was 44% (95% CI: 26.7, 60.3). Key Secondary endpoints: Responses were durable in de novo 1L BPDCN patients (PAP). Median duration of CR+CRc (DOCR) (months): 10.6 (95% CI: 3.8, NE (not estimable)). DOCR in #HSCT patients (months): NR (not reached) (95% CI: 4.4, NE). DOCR in non-HSCT patients (months): 6.8 (95% CI: 1.6, 9.8). Similar CR+CRc rate across all 1L BPDCN (de novo+PCHM (pre-existing or coexisting hematologic malignancy), n=33): 69.7% (95% CI: 51.3, 84.4). Median DOCR: 9.8 months (95% CI: 4.6, NE). CR+CRc rate in R/R BPDCN (n=51) was at 13.7% (95% CI: 5.7, 26.3); Median DOCR: 9.2 months (95% CI: 2.4, NE).

TABLE 13
Efficacy and Durability Evident Across Primary
Endpoint and Key Secondary Endpoints

| | PAP (Frontline | | Total Frontline | |
|---|---|---|---|---|
| | BPDCN | | BPDCN | |
| | Patients) | | patients | |
| | (N = 20) | | (N = 33) | |
| CR/CRc rate, n (%) | 15 | (75.0) | 23 | (69.7) |
| 95% CI | 50.90, 91.34 | | 51.29, 84.41 | |
| Duration of CR/CRc (months) | | | | |
| Median (95% CI) | 10.6 | (3.8, NE) | 9.8 | (4.6, NE) |
| Min, Max | 1.6, 23.9+ | | 1.6, 23.9+ | |
| HSCT in CR/CRc, n/N (%) | 8/15 | (53.3) | 12/23 | (52.2) |
| Duration of CR/CRc in HSCT | | | | |
| patients (months) | | | | |
| Median (95% CI) | NR | (4.4, NE) | NR | (4.4, NE) |
| Min, Max | 4.4+, 23.9+ | | 3.2, 23.9+ | |
| Duration of CR/CRc in non- | | | | |
| HSCT patients (months) | | | | |
| Median (95% CI) | 6.8 | (1.6, 9.8) | 6.8 | (1.9, 9.2) |

| | | |
|---|---|---|
| Min, Max | 1.6, 19.0+ | 1.6, 19.0+ |

| | R/R BPDCN | |
|---|---|---|
| | Patients | |
| | (N = 51) | |
| CR/CRc rate, n (%) | 7 | (13.7) |
| 95% CI | 5.70, 26.26 | |
| Duration of CR/CRc (months) | | |
| Median (95% CI) | 9.2 | (2.4, NE) |
| Min, Max | 2.4, 27.6+ | |
| HSCT in CR/CRc, n/N (%) | 2/7 | (28.6) |
| Duration of CR/CRc in HSCT | | |
| patients (months) | | |
| Median (95% CI) | NR | (NE, NE) |
| Min, Max | 4.1, 12.4+ | |
| Duration of CR/CRc in non-HSCT | | |
| patients (months) | | |
| Median (95% CI) | 9.2 | (2.4, NE) |
| Min, Max | 2.4, 27.6+ | |
| CR/CRc rate in prior tagraxofusp, n (%) | 4/29 | (13.8) |
| 95% CI | 3.9, 31.7 | |
| Duration of CR/CRc in prior | | |
| tagraxofusp (months) | | |
| Median (95% CI) | NR | (9.2, NE) |

| | |
|---|---|
| Min, Max | 4.1, 12.4+ |

(NA: Not Applicable; #NR/NE: not reached/not estimable; += censored at cutoff)

Baseline and efficacy summaries from pivekimab sunirine and Elzonris monotherapy in BPDCN patients is shown in FIG. 8.

It is to be appreciated that the Detailed Description section, and not the Summary and Abstract sections, is intended to be used to interpret the claims. The Summary and Abstract sections sets forth one or more, but not all, exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1.-141. (canceled)

142. A method of treating an adult human patient with blastic plasmacytoid dendritic cell neoplasm (BPDCN), the method comprising administering to the patient by infusion a safe and effective dose of about 0.045 mg/kg of pivekimab sunirine once in a 21-day cycle, wherein the patient is administered one or more cycles of infusion, and wherein the pivekimab sunirine is administered by a controlled rate of infusion.

143. The method of claim 142, wherein the pivekimab sunirine is administered in an infusion having a duration of no more than 30 minutes.

144. The method of claim 142, wherein the pivekimab sunirine is administered in an infusion having a duration of about 15 to 30 minutes.

145. The method of claim 142, wherein the controlled rate of infusion is from about 0.8 mL/min to about 1.7 mL/min.

146. The method of claim 142, wherein the controlled rate of infusion is about 0.8 mL/min (about 50 mL/hour or about 0.165 mg/min) for the first 30 minutes.

147. The method of claim 146, wherein the controlled rate of infusion is increased to about 1.7 mL/min.

148. The method of claim 142, wherein the patient has a history of central nervous system (CNS) involvement in the BPDCN.

149. The method of claim 142, wherein the pivekimab sunirine is further administered as a maintenance therapy.

150. The method of claim 149, wherein the maintenance therapy comprises administering the pivekimab sunirine once in a 21-day cycle.

151. The method of claim 142, wherein CD123 appears in a sample obtained from the patient prior to the administration of the pivekimab sunirine.

152. The method of claim 142, wherein the patient has an absolute neutrophil count of greater than 500/μL.

153. The method of claim 142, wherein the patient has been pretreated with a premedication prior to administration of the pivekimab sunirine, optionally wherein the premedication is a corticosteroid, diphenhydramine, acetaminophen, paracetamol, or a combination thereof.

154. The method of claim 142, further comprising pretreating the patient with a premedication prior to administration of the pivekimab sunirine, optionally wherein the premedication is a corticosteroid, an antihistamine, an antipyretic, or a combination thereof.

155. The method of claim 154, wherein the corticosteroid is administered to the patient on the day prior to administering the pivekimab sunirine and on the same day as administering the pivekimab sunirine, to the patient.

156. The method of claim 154, wherein the corticosteroid is administered to the patient twice on the day prior to administering the pivekimab sunirine and on the same day as administering the pivekimab sunirine, to the patient.

157. The method of claim 154, wherein the corticosteroid is dexamethasone.

158. The method of claim 157, wherein the dexamethasone is administered at a dose of 8 mg or 10 mg.

159. The method of claim 154, wherein the patient is further premedicated with an antihistamine and an antipyretic at least 30 minutes prior to administering the pivekimab sunirine.

160. The method of claim 159, wherein the antihistamine is diphenhydramine and the antipyretic is acetaminophen or paracetamol.

161. The method of claim 160, wherein diphenhydramine is administered intravenously at a dose of 25 mg to 50 mg; and (i) acetaminophen is administered orally or intravenously at a dose of 325 mg to 650 mg or (ii) paracetamol is administered orally or intravenously at a dose of 500 mg to 1000 mg.

162. The method of claim 142, wherein the BPDCN is frontline BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 5 months of the first administration, and wherein the duration of the CR, CRc, CRh, or CRi is at least 12 months.

163. The method of claim 142, wherein the BPDCN is relapsed or refractory (R/R) BPDCN, and wherein the administration achieves a complete response (CR), CR (clinical) with minimal residual skin abnormality (CRc), CR (clinical) with partial hematological recovery (CRh), or CR (clinical) with incomplete recovery (CRi) within 2 months of the first administration, and wherein the duration of the CR, CRc, CRh, or CRi is at least 6 months or at least 7 months.

164. The method of claim 142, wherein the BPDCN is frontline BPDCN, and wherein the administration achieves a complete response (CR) or CR (clinical) with minimal residual skin abnormality (CRc), and wherein the duration of the CR or CRc is at least 9 months.

165. The method of claim 142, wherein the BPDCN is R/R BPDCN, and wherein the administration achieves a complete response (CR) or CR (clinical) with minimal residual skin abnormality (CRc), and wherein the duration of the CR or CRc is at least 9 months.

166. A method of preparing an adult human patient with blastic plasmacytoid dendritic cell neoplasm (BPDCN) for hematopoietic stem cell transplant (HSCT), the method comprising administering to the patient, by infusion, a safe and effective dose of about 0.045 mg/kg of pivekimab sunirine once in a 21-day cycle, wherein the patient is administered one or more cycles of infusion, wherein said administration results in a period of response or remission of the BPDCN sufficient for said patient to be administered HSCT.

167. The method of claim 166, wherein the administration of pivekimab sunirine results in the patient achieving a complete response (CR), CR (clinical) with minimal residual skin abnormality (CRc), CR (clinical) with partial hematological recovery (CRh), CR (clinical) with incomplete recovery (CRi), or a partial remission/partial response (PR), wherein said CR, CRc, CRh, CRi or PR comprises a hematologic recovery comprising a neutrophil count of greater than or equal to 500 neutrophils/microliter and a platelet count of greater than or equal to 50,000 platelets/microliter.

168. The method of claim 166, wherein a corticosteroid is administered to the patient on the day prior and once prior to and on the same day as administering the pivekimab sunirine to the patient.

169. The method of claim 166, wherein a corticosteroid is administered to the patient twice on the day prior and once prior to and on the same day as administering the pivekimab sunirine to the patient.

170. The method of claim 168, wherein the corticosteroid is dexamethasone.

171. The method of claim 170, wherein the dexamethasone is administered at a dose of 8 mg or 10 mg.

172. The method of claim 168, wherein the patient is further premedicated with an antihistamine and an antipyretic at least 30 minutes prior to administering the pivekimab sunirine.

173. The method of claim 172, wherein the antihistamine is diphenhydramine and the antipyretic is acetaminophen or paracetamol.

174. The method of claim 173, wherein diphenhydramine is administered intravenously at a dose of 25 mg to 50 mg; and (i) acetaminophen is administered orally or intravenously at a dose of 325 mg to 650 mg; or (ii) paracetamol is administered orally or intravenously at a dose of 500 mg to 1000 mg.

175. The method of claim 166, wherein the pivekimab sunirine is administered by a controlled rate of infusion.

176. The method of claim 175, wherein the pivekimab sunirine is administered by the controlled rate of infusion having a duration of no more than 30 minutes.

177. The method of claim 175, wherein the pivekimab sunirine is administered by the controlled rate of infusion having a duration of about 15 to 30 minutes.

178. The method of claim 175, wherein the controlled rate of infusion is from about 0.8 mL/min to about 1.7 mL/min.

179. The method of claim 175, wherein the controlled rate of infusion is about 0.8 mL/min (about 50 mL/hour or about 0.165 mg/min) for the first 30 minutes.

180. The method of claim 175, wherein the controlled rate of infusion is increased to about 1.7 mL/min (about 100 mL/hour or about 0.33 mg/min).

181. The method of claim 166, wherein the HSCT is administered about 4 to about 8 weeks after the administration of the pivekimab sunirine.

182. The method of claim 166, wherein the patient has received tagraxofusp prior to administering the pivekimab sunirine.

183. The method of claim 166, wherein the patient has previously been treated with venetoclax; a hypomethylating agent; cyclophosphamide, vincristine sulfate, and prednisone (CVP); steroids; or combinations thereof.

184. The method of claim 166, wherein the patient has previously been treated with cyclophosphamide, vincristine sulfate, doxorubicin hydrochloride, methotrexate, cytarabine, and dexamethasone (HyperCVAD); fludarabine, high-dose cytarabine, and G-CSF (FLAG); and/or cyclophosphamide, vincristine and doxorubicin (CHOP).

185. The method of claim 166, wherein the BPDCN is relapsed or refractory (R/R) BPDCN.

186. The method of claim 166, wherein the patient has received 1-3 prior systemic therapies.

187. The method of claim 166, wherein the patient has a prior or concomitant hematological malignancy (PCHM).

188. The method of claim 166, wherein the patient has liver enzymes less than or equal to 2.5× the upper limit of normal, total bilirubin less than or equal to 1.5× the upper limit of normal, glomerular filtration rate of greater than 30 mL/min/1.73 m² or creatinine clearance of greater than 30 mL/min, and left ventricular ejection fraction of greater than or equal to 45%.

189. A method of treating an adult human patient with blastic plasmacytoid dendritic cell neoplasm (BPDCN), the method comprising:
on a first day, administering a corticosteroid to the patient;
on a second day, administering the corticosteroid prior to a safe and effective infused dose of about 0.045 mg/kg of pivekimab sunirine.

190. The method of claim 189, wherein the corticosteroid is dexamethasone, administered at a dose of 8 mg or 10 mg.

191. The method of claim 189, wherein the corticosteroid is administered twice on the first day, and wherein the corticosteroid is administered once on the second day.

192. The method of claim 189, wherein on the second day, the corticosteroid is administered at least 30 minutes prior to the safe and effective infused dose of pivekimab sunirine.

193. The method of claim 189, further comprising administering an antihistamine and an antipyretic on the second day prior to the safe and effective infused dose of pivekimab sunirine.

194. The method of claim 193, wherein the antihistamine and the antipyretic are administered at least 30 minutes prior to the safe and effective infused dose of pivekimab sunirine.

195. The method of claim 193, wherein the antipyretic is acetaminophen, administered at a dose of about 325 mg to about 650 mg.

196. The method of claim 193, wherein the antipyretic is paracetamol, administered at a dose of about 500 mg to about 1000 mg.

197. The method of claim 193, wherein the antihistamine is diphenhydramine, administered at a dose of about 25 mg to about 50 mg.
